(19) United States
(12) Patent Application Publication
(10) Pub. No.: US 2026/0223835 A1
(43) Pub. Date: Aug. 6, 2026

(54) COMPOSITION AND METHOD FOR CRYOPRESERVATION OF CELLS

(71) Applicant: Regents of the University of Minnesota, Minneapolis, MN (US)

(72) Inventors: Rui Li, Minneapolis, MN (US); Allison Hubel, Minneapolis, MN (US)

(73) Assignee: Regents of the University of Minnesota, Minneapolis, MN (US)

(21) Appl. No.: 19/634,563

(22) Filed: Mar. 31, 2026

Related U.S. Application Data
(63) Continuation of application No. 17/607,695, filed on Apr. 24, 2020.
(60) Provisional application No. 62/840,617, filed on Apr. 30, 2019.

(51) Int. Cl. A01N 1/125; A61K 47/26; C07K 14/76

(52) U.S. Cl. CPC A01N 1/125; A61K 47/26; C07K 14/76

(57) ABSTRACT

A cryopreservative composition includes a sugar component with a total concentration of sugar components in the composition of 300 mM or less; a sugar alcohol component, with a total concentration of sugar alcohol components in the composition of 2 M or less; and at least one of a polymer component and albumin, with the proviso that the composition includes less than a cryopreservative amount of dimethyl sulfoxide (DMSO). A method of cryopreserving a cell includes adding a cell to the cryopreservative composition; freezing the composition; storing the frozen composition; thawing the composition; removing the cell from the thawed composition; and culturing the cell under conditions effective for the cell to remain viable. The freezing may include cooling at a rate of 0.1° C./min to 5° C./min. The method may be performed without a washing step after thawing.

### CONTINUING APPLICATION DATA

This is a continuation of co-pending application Ser. No. 17/607,695, filed Oct. 29, 2021, which is a national stage entry of PCT application Serial No. PCT/US20/29847, filed Apr. 24, 2020, which claims priority to U.S. provisional application Ser. No. 62/840,617, filed Apr. 30, 2019, all of which are incorporated herein by reference.

### GOVERNMENT FUNDING

This invention was made with government support under EB023880 awarded by the National Institutes of Health. The government has certain rights in the invention.

### FIELD

The present application relates to compositions and methods for cryopreserving cells.

### SUMMARY

The present application relates to compositions and methods for cryopreserving cells. The cryopreservative composition may include a sugar component with a total concentration of sugar components in the composition of 300 mM or less; a sugar alcohol component, with a total concentration of sugar alcohol components in the composition of 2 M or less; and at least one of a polymer component at a concentration of 1% to 15% and albumin at a concentration of 0.5% to 10%, with the proviso that the composition includes less than a cryopreservative amount of dimethyl sulfoxide (DMSO).

The sugar component may be provided at a concentration of 1 mM to 100 mM. The sugar component may include trehalose, maltose, lactose, fructose, sucrose, glucose, dextran, melezitose, raffinose, nigerotriose, maltotriose, maltotriulose, kestose, cellobiose, chitobiose, lactulose, or a combination thereof.

The sugar alcohol component may be provided at a concentration of 0.2 M to 1.2 M. The sugar alcohol component may include glycerol, sorbitol, ethylene glycol, propylene glycol, inositol, xylitol, mannitol, arabitol, ribitol, erythritol, threitol, galactitol, pinitol, or a combination thereof.

The cryopreservative composition may further include an ionic component at a concentration of 0.1% to 2.5%. The cryopreservative composition may further include an amino acid component at a concentration of 0.1 mM to 50 mM. The amino acid component may include isoleucine, creatine, or a combination thereof. The cryopreservative composition may further include a secondary amino acid component. The secondary amino acid component may include one or more proline, valine, alanine, glycine, asparagine, aspartic acid, glutamic acid, serine, histidine, cysteine, tryptophan, tyrosine, arginine, glutamine, taurine, betaine, ectoine dimethylglycine, ethylmethylglycine, an RGD peptide, or a combination thereof.

The cryopreservative composition may further include a cell. The cell may be an iPS cell. The cell may be a viable recovered cryopreserved cell.

A method of cryopreserving a cell includes adding a cell to the cryopreservative composition; freezing the composition; storing the frozen composition at a temperature below 0° C.; thawing the composition; removing the cell from the thawed composition; and culturing the cell under conditions effective for the cell to remain viable. The freezing of the composition comprises cooling at a rate of greater than 0° C./min and up to 5° C./min. The method may be performed without a washing step after thawing. The cryopreservation method of the present disclosure actively induces ice nucleation at a defined temperature.

### BRIEF DESCRIPTION OF DRAWINGS

FIGS. 1A-1E show results from the Examples.

FIGS. 2A-2D show results from the Examples.

FIGS. 3A-3C show results from the Examples.

FIGS. 4A-4C show cooling profiles used in the Examples.

FIG. 5 is a graphical representation of the cryopreservation workflow used in the Examples.

FIGS. 6A-6C show results from the Examples.

FIGS. 7A-7F show results from the Examples.

FIGS. 8A-8F show results from the Examples.

FIGS. 9A-9F show results from the Examples.

FIGS. 10A-10B show results from the Examples.

FIG. 11 shows results from the Examples.

FIG. 12 shows results from the Examples.

FIG. 13 shows results from the Examples.

FIG. 14 shows results from the Examples.

FIG. 15 shows results from the Examples.

### DETAILED DESCRIPTION

The present disclosure relates to compositions and methods for cryopreserving cells. In particular, the present disclosure relates to compositions and methods for cryopreserving cells without the use of DMSO. The compositions of the present disclosure can be used to cryopreserve cells in a manner that does not require washing the cells after cryopreservation and before subsequent use.

The term “substantially” as used here has the same meaning as “significantly,” and can be understood to modify the term that follows by at least about 75%, at least about 90%, at least about 95%, or at least about 98%. The term “not substantially” as used here has the same meaning as “not significantly,” and can be understood to have the inverse meaning of ‘substantially,” i.e., modifying the term that follows by not more than 25%, not more than 10%, not more than 5%, or not more than 2%.

The term “about” is used here in conjunction with numeric values to include normal variations in measurements as expected by persons skilled in the art, and is understood have the same meaning as “approximately” and to cover a typical margin of error, such as ±5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

The term “cryopreservative amount” is used here to refer to an amount that is sufficient to provide cryopreservation of a sample, such as cells. The phrase “less than a cryopreservative amount of dimethyl sulfoxide (DMSO)” may refer to less than 140 mM of DMSO.

Induced Pluripotent Stem Cells (iPS cells) are foundational to current approaches to regenerative medicine. For example, human induced pluripotent stem cells (hiPSCs) are multicellular aggregates that can be formed from reprogramming from a variety of somatic cells and have the potential to be differentiated into all three germ layers, attracting much interest in tissue engineering, disease modeling, and personalized medicine. iPS cells can be differentiated to form hepatocytes that function as liver cells, and can be used in liver assist devices or transplantable livers to treat liver failure. iPS cells can also be differentiated into neuronal cells, cardiac cells, and other cell types that are otherwise hard to obtain. However, the development of such therapies has been limited by cell availability.

For both clinical and scientific purposes, effective cryopreservation of iPS cells is required for transportation, storage of frozen iPS cells, and other downstream uses. However, cryopreserved iPS cells are vulnerable to loss of viability, function, or pluripotency.

Current practices at research laboratories and biorepositories involve cryopreserving iPS cells either as aggregates or single cells, depending on the desired use. Cryopreservation of iPS cells typically involves one of two types of solutions: DMSO-containing solutions or scarcely DMSO-free solutions; and one of two different methods: conventional slow cooling or vitrification. Vitrification typically uses high concentrations of cryoprotective agents combined with high cooling rates to avoid the formation of ice during freezing. For example, samples may be contained in specialized straws or membranes and be rapidly immersed in liquid nitrogen. In step-wise cooling, samples may be transferred from 0° C. (on ice) to −20° C., and then to −80° C. for non-standardized lengths of time, and finally stored at −196° C. Specialized containers are available (sold under trade names MR. FROSTY™ and COOLCELL™) that are constructed to contain the cells inside a freezer and to slow down the cooling rate to approximately 1° C./min. Controlled rate freezers are also available that provide a controlled freezing rate. Conventional slow cooling typically implies using a solution of 10% dimethyl sulfoxide (DMSO) and a cooling rate of 1° C./min. High rates of post-thaw recovery (up to about 100%) are observed with vitrification, but the method faces limitations of poor scalability and high risk of contamination.

DMSO is known to be toxic to cells and can alter the epigenetics of the cells. For cells that are reprogrammed, such as iPS cells, this can lead to concerns for the downstream use of the cells. Specifically, one of the concerns with iPS cells is genetic stability. Factors that influence genetic stability cause concerns regarding clinical use of the cells.

Using DMSO in the cryopreserving solution forces users to freeze the cells quickly after introduction of the solution and to wash the solution off quickly after thawing to minimize exposure time of unfrozen/thawed cells to DMSO. This requirement influences workflow, making it more difficult and more expensive to preserve the cells.

Cell banks that store iPS cells describe high variability in the cell viability. It is common for one vial of a given cell line to exhibit relatively high viability, and another vial of the same cell line to exhibit very poor viability. The variability in outcomes makes it more costly and difficult to use iPS cells for research and potential cell therapy manufacture.

Despite attempts to improve cryopreservation of multicellular aggregates, the mechanisms of damage from different freezing conditions are poorly understood. As with single cells, extensive intracellular ice formation (IIF) has been proven to be damaging. Exposure to high solute concentration at low temperatures can cause a solute effect in cells, and the addition or removal of cryoprotectants can result in cell losses resulting from osmotic stress. However, freezing responses of cell aggregates are more complex than those in single cells. For example, propagation of IIF in cell aggregates through gap junctions has been observed experimentally in a variety of cell types.

Rho-kinase (“ROCK”) inhibitor (ROCKi) Y-27632 has been used to enhance the survival of dissociated hiPSCs (that is, single cells). Disruption of nonmuscle myosin IIA (NMMIIA) and actin as a result of ROCK inhibition has been shown to increase survival and pluripotency of single hiPSCs. However, the addition of ROCKi can have contradictory effects in the case of hiPSC aggregates. The downregulation of NMMIIA has been shown to impair cell adhesion, cell-cell junctions, self-renewal, and pluripotency of hiPSC aggregates.

Distinct from common practice in stem cell research, which uses NALGENE® MR. FROSTY™ (ThermoFisher Scientific) or equivalent freezing containers in an ultra-low freezer that results in passive freezing with spontaneous and variable cooling profile, the cryopreservation method of the present disclosure uses controlled-rate freezing, which is programmed to follow a defined and consistent cooling profile. Also distinct from existing practice in research laboratories and stem cell repositories that uses controlled-rate freezing with defined cooling rate but spontaneous and variable ice nucleation temperature, the cryopreservation method of the present disclosure actively induces ice nucleation at a defined temperature. The cooling profile (cooling rate at high subzero temperatures and the nucleation temperature) affects the quality of cryopreserved cells. Variability in the cooling profile contributes to variability in survival and function of cryopreserved cells. Suboptimal cooling rates and ice nucleation temperatures are likely when they are allowed to occur spontaneously, which leads to severe cell death and failed post-thaw culture, a common experience among users of cryopreserved iPS cells under existing practices. In comparison, the cryopreservation method of the present disclosure defines both the ice nucleation temperature as well as the cooling rate of the cooling profile, which minimizes variability and improves quality of cryopreserved cells.

Cryopreservation compositions and methods that are simple to use, result in high cell viability, and/or do not require the use of ROCK inhibitors are desired.

The cryopreservation compositions and methods of the present disclosure are suitable for use with various types of cells and cell formations. For example, the cryopreservation compositions and methods may be used with dissociated single cells in suspension; dissociated 2-dimensional clusters of cells with intact cell-cell adhesion, for example suspended in an aqueous solution; 3-dimensional aggregates of cells, for example suspended in an aqueous solution; single cells embedded in a 3-dimensional matrix (e.g., a hydrogel); 2-dimensional monolayer of cells adhered on the surface of a 3-dimensional matrix (e.g., a hydrogel); 2-dimensional monolayer of cells adhered on the surface of a 2-dimensional matrix; and organoids.

The cryopreservation compositions and methods of the present disclosure are suitable for cryopreserving iPS and other types of cells. Various cell types may include, for example, an iPS cell, an embryonic stem cell, or an embryoid body; a hepatocyte or a liver organoid; a neuron or a neural progenitor cell, or a neurosphere or a brain organoid; a glial cell or a glial progenitor cell; a cardiomyocyte, a cardiac progenitor cell, cardiac tissue, or a cardiac organoid; an endothelial cell or an endothelium; an epithelial cell or an epithelium; a myocyte, a smooth muscle cell, skeletal muscle tissue, or smooth muscle tissue; a tenocyte or tendon tissue; an osteocyte, a chondrocyte, or an osteochondroprogenitor cell; a beta cell, pancreatic islet tissue, or a pancreatic organoid; an adipocyte or adipose tissue; a corneal cell or a cornea; a retinal cell or retinal tissue; a trabecular meshwork cell or trabecular meshwork tissue; an intestinal cell, intestinal tissue, or an intestinal organoid; a renal cell or a kidney organoid; a hematopoietic cell; a vascular cell, a lymphatic cell, a blood vessel, or a lymphatic vessel; or a gamete.

Cryopreservation of a cell, cells, or tissue may include cooling and/or storing the specimen. The specimen may be cooled to or stored at a temperature 0° C. or lower, −20° C. or lower, −40° C. or lower, −90° C. or lower, −150° C. or lower, −190° C. or lower, or −196° C. or lower. The specimen may be cooled to or stored at a temperature below 0° C. and at −196° C. or higher. For example, the specimen may be cooled to or stored at a temperature ranging from 0° C. to −196° C., from 0° C. to −40° C., from −40° C. to −196° C., from −40° C. to −90° C., or from −90° C. to −196° C.

According to an embodiment, the method of the present disclosure may be used to store samples at relatively high subzero temperatures, such as between 0° C. and −40° C. Thus, distinct from the final temperatures of conventional cryopreservation methods (from −90° C. to −196° C.) using a controlled-rate freezer, in some embodiments the final temperature of a freezing method of the present disclosure may be closer to the temperature of a common household or laboratory freezer, between −18° C. and −30° C. Accordingly, in some embodiments the method of the present disclosure does not require the use of specialized equipment, which may include a controlled-rate freezer and a cryogenic storage unit, or specialized materials, which may include liquid nitrogen, or specialized handling procedures, which may include transferring frozen samples from the freezing modality to the storage unit. Distinct from the range of sample stability targeted by liquid nitrogen storage, which may be years, or decades, some embodiments of the method of the present disclosure may target a range of sample stability in high sub-zero storage of days, or weeks, or months. In other embodiments, the method of the present disclosure targets long range storage stability, such as years or decades.

According to some embodiments of the present disclosure, the cryopreservation composition is free of or substantially free of DMSO. According to an embodiment, the cryopreservation composition of the present disclosure can be used without washing the cells after thawing. Further according to an embodiment, the cryopreservation composition and the cryopreservation method of the present disclosure results in one or more of high recovery, high viability, normal growth rate, and normal karyotype of thawed cells. Cell survival may be maintained withing certain ranges of concentrations and other parameters as discussed within this disclosure. For example, cell survival may be maintained while varying the time of cell exposure to the composition before freezing; the cooling rate; the ice nucleation temperature; the time of cell exposure to the composition after thawing; or any combination thereof. The cryopreservation methods of the present disclosure may include multiple cycles of freezing, thawing, and culturing. The cryopreservation methods of the present disclosure may be executed by an automated cell culture system.

According to an embodiment, the cryopreservation composition includes at least one primary component and at least one secondary component. The primary components may include a sugar component, a sugar alcohol component, an amino acid component, a polymer component, a protein component, or a combination thereof. In some embodiments, the primary components include at least a sugar component and a sugar alcohol component. In some embodiments, the primary components include a sugar component, a sugar alcohol component, an amino acid component, a polymer component, and a protein component. The amino acid component may be further divided into a primary amino acid component and a secondary amino acid component. In some embodiments, the composition includes both a primary and a secondary amino acid component. In some embodiments, the composition includes only a primary amino acid component or a secondary amino acid component.

The sugar component may include any suitable mono-, di-, or trisaccharide or a combination thereof. For example, the sugar component may include trehalose, maltose, lactose, fructose, sucrose, glucose, dextran, melezitose, raffinose, nigerotriose, maltotriose, maltotriulose, kestose, cellobiose, chitobiose, lactulose, or a combination of sugars. The sugar component may have a concentration of at least 1 mM such as, for example, at least 2 mM, at least 3 mM, at least 4 mM, at least 5 mM, at least 6 mM, at least 7 mM, at least 8 mM, at least 9 mM, at least 10 mM, at least 20 mM, at least 30 mM, at least 40 mM, at least 50 mM, at least 100 mM, at least 150 mM, at least 200 mM, or at least 250 mM. The sugar component may be provided at a maximum concentration of (e.g., the total amount of sugars is) no more than 500 mM such as, for example, no more than 400 mM, no more than 300 mM, no more than 250 mM, no more than 200 mM, no more than 150 mM, no more than 125 mM, no more than 100 mM, no more than 90 mM, no more than 80 mM, no more than 70 mM, no more than 60 mM, or no more than 50 mM. The sugar component may be provided at a concentration within a range having endpoints defined by any minimum concentration listed above and any maximum concentration listed above that is greater than the minimum concentration. When more than one sugar is present in the composition, the concentration of the sugar component reflects the total concentration of all sugars in the composition. Thus, in some embodiments, the sugar component may be present at a concentration of from 0.1 mM to 250 mM such as, for example, from 1 mM to 250 mM, from 1 mM to 200 mM, from 2 mM to 150 mM, from 5 to 120 mM, from 10 mM to 100 mM, from 15 to 100 mM, or from 20 mM to 80 mM. In some embodiments, the sugar component includes from 10 mM to 200 mM, from 20 to 120 mM, or from 30 mM to 80 mM of trehalose, maltose, lactose, or a combination thereof.

The sugar alcohol component may include any suitable sugar alcohols or a combination thereof. For example, the sugar alcohol component can include glycerol, sorbitol, ethylene glycol, propylene glycol, inositol, xylitol, mannitol, arabitol, ribitol, erythritol, threitol, galactitol, pinitol, or a combination of sugar alcohols. The sugar alcohol component may have a concentration of at least 0.1 M such as, for example, at least 0.2 M, at least 0.3 M, at least 0.4 M, at least 0.5 M, at least 0.6 M, at least 0.7 M, at least 0.8 M, at least 0.9 M, or at least 1.0 M. The sugar alcohol component may be provided at a maximum concentration of (e.g., the total amount of sugar alcohols is) no more than 2.0 M such as, for example, no more than 1.9 M, no more than 1.8 M, no more than 1.7 M, no more than 1.6 M, no more than 1.5 M, no more than 1.4 M, no more than 1.3 M, no more than 1.0 M, no more than 0.90 M, no more than 0.8 M, no more than 0.7 M, no more than 0.6 M, or no more than 0.5 M. The sugar alcohol component may be provided at a concentration within a range having endpoints defined by any minimum concentration listed above and any maximum concentration listed above that is greater than the minimum concentration. When more than one sugar alcohol is present in the composition, the concentration of the sugar alcohol component reflects the total concentration of all sugar alcohols in the composition. Thus, in some embodiments, the sugar alcohol component may be present at a concentration of 0.1 M to 1.2 M, from 0.2 M to 1.2 M, from 0.2 M to 1 M, from 0.3 M to 1 M, from 0.5 M to 1 M, or from 0.3 M to 0.8 M. For example, certain embodiments can include glycerol at a concentration of 0.2 M to 1.0 M. Other particular embodiments can include an alternative sugar alcohol at a concentration of 0.3 M to 0.8 M.

The amino acid component may include any suitable amino acids, amino acid derivatives, peptides, or a combination thereof. For example, the amino acid component may include isoleucine (e.g., L-isoleucine), proline (e.g., L-proline), valine (e.g., L-valine), alanine (e.g., L-alanine), glycine, asparagine (e.g., L-asparagine), aspartic acid (e.g., L-aspartic acid), glutamic acid (e.g., L-glutamic acid), serine (e.g., L-serine), histidine (e.g., L-histidine), cysteine (e.g., L-cysteine), tryptophan (e.g., L-tryptophan), tyrosine (e.g., L-tyrosine), arginine (e.g., L-arginine), glutamine (e.g., L-glutamine), creatine (e.g., L-creatine), taurine (e.g., L-taurine), betaine, ectoine dimethylglycine, ethylmethylglycine, an RGD peptide, or a combination thereof. Of the amino acids, isoleucine and creatine may be considered primary amino acid components. The amino acid component may have a concentration of at least 0.1 mM such as, for example, at least 1 mM, at least 2 mM, at least 3 mM, at least 4 mM, at least 5 mM, at least 6 mM, at least 7 mM, at least 8 mM, at least 9 mM, or at least 10 mM. The amino acid component may be provided at a maximum concentration of no more than 100 mM such as, for example, no more than 80 mM, no more than 50 mM, no more than 40 mM, no more than 30 mM, no more than 25 mM, no more than 22.5 mM, no more than 20 mM, no more than 15 mM, no more than 14 mM, or no more than 10 mM. The concentration of the amino acid component may refer to the amino acids as a whole (including primary and secondary), or to the primary amino acid component alone. Thus, in some embodiments, the amino acid component may be present at a concentration of 0 mM to 80 mM, from 0.1 mM to 50 mM, from 1 mM to 15 mM, from 1 mM to 10 mM, or from 2 mM to 10 mM. For example, certain embodiments can include the amino acid component at a concentration of 1 mM to 15 mM. Other particular embodiments can include the amino acid component at a concentration of 2 mM to 10 mM.

The polymer component may include any suitable polymer components or a combination thereof. According to an embodiment, suitable polymer components are biocompatible, hydrophilic or amphiphilic, non-cell adhesion molecule (“CAM”)-binding polymers. For example, the polymer component may include poloxamer (e.g., poloxamer 142, poloxamer 188, poloxamer 331, or poloxamer 407), alginate, polyethylene glycol, polyglutamic acid, polyvinyl alcohol, polyvinyl pyrrolidone, or a combination thereof. The polymer component may have a concentration of at least 1% (w/v), at least 2%, at least 3%, at least 4%, or at least 5%. The polymer component may be provided at a maximum concentration of no more than 15%, no more than 12%, no more than 10%, no more than 8%, no more than 7%, no more than 6%, or no more than 5%. Thus, in some embodiments, the polymer component may be present at a concentration of 1% to 15%, from 1% to 12%, from 2% to 12%, from 1% to 10%, from 3% to 10%, or from 3% to 8%. For example, certain embodiments can include the polymer component at a concentration of 2% to 12%. Other particular embodiments can include the polymer component at a concentration of 3% to 8%.

The protein component may include any suitable serum constituents or a combination thereof. For example, the protein component may include albumin. The protein component may have a concentration of at least 0.5% (w/v), at least 1%, at least 1.5%, at least 2%, or at least 3%. The protein component may be provided at a maximum concentration of no more than 10%, no more than 8%, no more than 6%, no more than 5%, no more than 4%, no more than 3%, or no more than 2%. Thus, in some embodiments, the protein component may be present at a concentration of 0% to 10%, from 0.5% to 10%, from 0% to 6%, from 1% to 6%, from 0% to 5%, from 1% to 5%, or from 1.5% to 4%. For example, certain embodiments can include the protein component at a concentration of 1% to 6%. Other particular embodiments can include the protein component at a concentration of 1.5% to 4%.

The secondary components may include one or more ionic components, such as salts, inorganic ions, pH balancing agents, and combinations thereof. The secondary amino acid component may also be considered one of the secondary components. In some embodiments, the secondary components include at least a salt, an inorganic ion, a pH balancing agent, and a secondary amino acid component.

The ionic component may include any suitable ionic compounds or a combination thereof. For example, the ionic component may include salts, acids, or bases that provide ions such as Ca⁺, Mg²⁺, Na⁺, K⁺, Cl⁻, HCO³⁻, or the like, or a combination thereof. Examples of suitable salts include CaCl₂, MgCl₂, MgSO₄, KCl, KH₂PO₄, NaHCO₃, NaCl, Na₂HPO₄, and the like. The ionic component may have a concentration of at least 0.05% (w/v), at least 0.1%, at least 0.2%, at least 0.3%, at least 0.4%, at least 0.5%, at least 0.6%, or at least 0.7%. The ionic component may be provided at a maximum concentration of no more than 2.5% (w/v), no more than 2%, no more than 1.5%, no more than 1.3%, no more than 1.2%, no more than 1.1%, or no more than 1.0%. Thus, in some embodiments, the ionic component may be present at a concentration of 0.1% to 2.5%. For example, certain embodiments can include the ionic component at a concentration of 0.2% to 2%. Other particular embodiments can include the ionic component at a concentration of 0.3% to 1.6%. The concentration of the ionic component may be expressed in molarity, and may range, for example, from 50 mM to 300 mM, from 75 mM to 250 mM, or from 100 mM to 200 mM.

The secondary amino acid component may include proteinogenic amino acids, non-proteinogenic amino acids, amino acid derivatives, or peptides. For example, the secondary amino acid component may include proline (e.g., L-proline), valine (e.g., L-valine), alanine (e.g., L-alanine), glycine, asparagine (e.g., L-asparagine), aspartic acid (e.g., L-aspartic acid), glutamic acid (e.g., L-glutamic acid), serine (e.g., L-serine), histidine (e.g., L-histidine), cysteine (e.g., L-cysteine), tryptophan (e.g., L-tryptophan), tyrosine (e.g., L-tyrosine), arginine (e.g., L-arginine), glutamine (e.g., L-glutamine), taurine (e.g., L-taurine), betaine, ectoine, dimethylglycine, ethylmethylglycine, an RGD peptide, or a combination thereof. The secondary amino acid components may have a concentration, either individually or in total, of at least 0.05 mM such as, for example, at least 0.08 mM, at least 0.1 mM, at least 0.12 mM, at least 0.15 mM, at least 0.2 mM, at least 0.4 mM, at least 0.6 mM, at least 0.8 mM, or at least 1 mM. The secondary amino acid components may be provided, either individually or in total, at a maximum concentration of no more than 10 mM such as, for example, no more than 8 mM, no more than 5 mM, no more than 4 mM, no more than 3 mM, no more than 2 mM, no more than 1.5 mM, no more than 1.3 mM, or no more than 1.0 mM. Thus, the secondary amino acid components may have a concentration, either individually or in total, ranging from 0.05 mM to 5 mM, 0.08 mM to 3 mM, or from 0.1 mM to 1.5 mM.

Generally, the composition can be free of DMSO or at least substantially free of DMSO. As used herein, “free of DMSO” refers to a composition that contains no more than trace amounts of DMSO and may be absolutely free of DMSO. As used herein, “at least substantially free of DMSO” refers to a solution that contains a level of DMSO that provides no greater cryopreservation than the remaining components of the solution—i.e., an amount of DMSO that is inconsequential to the functionality of the solution. Typical cryopreservative solutions include 10% DMSO. However, in some embodiments of the present disclosure, the amount of DMSO in the composition is less than 5% (w/v), less than 2%, less than 1%, or less than 0.1%, or is 0%.

Thus, in one aspect, this disclosure describes a cryopreservative composition. Generally, the cryopreservative composition includes a sugar component and a sugar alcohol component, as set forth in more detail above. In some embodiments, at least a portion of the sugar component may not necessarily penetrate the cell membrane and, therefore, acts on the outer surface of the cell. In such cases, the sugar component can include trehalose. In some embodiments, the cryopreservative composition can further include a polymer component, at least one amino acid, an ionic component, and albumin. Generally, the cryopreservative composition possesses an amount of DMSO that provides no more cryoprotection than the remaining components of the composition without the DMSO. An exemplary cryopreservative composition is given below.

| Component | Concentration/molarity |
|---|---|
| Sugar component (e.g., sucrose, trehalose, and dextrose) | 20-150 mM |
| Sugar alcohol component (e.g., glycerol) | 0.5-1 M |
| Amino acid component (e.g., L-isoleucine, proline, alanine, | 0-80 mM |
| glycine, asparagine, aspartic acid, glutamic acid, and serine) | |
| Polymer component (e.g., poloxamer) | 1-10% w/v |
| Albumin (e.g. human serum albumin, bovine serum albumin, | 0-5% w/v |
| and recombinant human albumin) | |
| Ionic component (mixture of salts including, e.g., CaCl₂, | 100-200 mM |
| MgCl₂, MgSO₄, KCl, KH₂PO₄, NaHCO₃, NaCl, Na₂HPO₄) | |

In some cases, the cryopreservative composition further includes a cell. Initially, the cell may be added to the cryopreservative composition prior to being cryopreserved and stored. In other cases, the cell may be being stored as a component of a frozen cryopreservative composition. In still other cases, the cell may be a viable cell recoverable from a thawed cryopreservative composition. As used herein, a “viable” cell includes a cell that remains living-under culture conditions suitable for the cell-after having been stored frozen in a cryoprotective solution, stored below 0° C., then thawed and removed from the cryoprotective composition. According to an embodiment, the cell does not need to be washed after thawing to remain viable.

This disclosure also describes a method of cryopreserving and storing a cell. The cell may be any live cell desired to be cryopreserved. In some embodiments the cell is a stem cell, such as an iPS cell. Generally, the method includes adding a cell to any embodiment of the cryoprotective composition described above, freezing the composition, storing the frozen composition at a temperature below 0° C., thawing the composition, removing the cell from the thawed composition, and culturing the cell under conditions effective for the cell to remain viable.

In some embodiments, the method includes controlled rates of cooling and/or controlled rates of re-warming. In some embodiments, the method includes initiating crystallization of molecular components in the cryopreservation composition at a temperature (referred to as the “ice nucleation temperature”) of 0° C. to −3° C., −1° C. to −20° C., −1° C. to −12° C., −12° C. to −20° C., −6° C. to −12° C., −1° C. to −8° C., −1.5° C. to −7° C., −2° C. to −6° C., −3° C. to −6° C., or −3° C. to −5° C. In one particular embodiment, the method includes initiating crystallization of molecular components in the cryopreservation composition at a temperature of about −4° C. The method may include cooling the cryoprotective composition with the cell at a rate greater than 0° C./min, such as 0.1° C./min or greater, 0.2° C./min or greater, 0.3° C./min or greater, 0.4° C./min or greater, 0.5° C./min or greater, 0.8° C./min or greater, 1.0° C./min or greater, 1.2° C./min or greater, 2° C./min or greater, 5° C./min or greater, or 10° C./min or greater. The method may include cooling the cryoprotective composition with the cell at a rate of 50° C./min or less, 20° C./min or less, 10° C./min or less, 5° C./min or less, 3° C./min or less, 2.5° C./min or less, 2.0° C./min or less, 1.8° C./min or less, 1.5° C./min or less, 1.2° C./min or less, or 1.0° C./min or less. Thus, the cryoprotective composition with the cell may be cooled at a rate of greater than 0° C./min and up to 5° C./min, 0.3° C./min to 3° C./min, 0.40° C./min to 2° C./min, 0.5° C./min to 1.5° C./min, or 0.8° C./min to 1.2° C./min. In one embodiment, the cryoprotective composition with the cell is cooled at a rate of about 1° C./min. The cryoprotective composition with the cell may be cooled to a typical storage temperature, such as about −5° C. or colder, −10° C. or colder, −20° C. or colder, −50° C. or colder, −70° C. or colder, −80° C. or colder, −100° C. or colder, −15° C. or colder, or −190° C. or colder. The cooling rate specified above may be maintained until the temperature is about −5° C. or colder, −10° C. or colder, −20° C. or colder, −25° C. or colder, −40° C. or colder, −50° C. or colder, −60° C. or colder, or until the final storage temperature is reached. The cryoprotective composition with the cell may be cooled further at a faster rate after reaching a temperature of about −5° C. or colder, −10° C. or colder, −20° C. or colder, −25° C. or colder, −40° C. or colder, −50° C. or colder, or −60° C. or colder.

The cryoprotective composition with the cell may be thawed at a rate of 20° C./min or greater, 40° C./min or greater, or 60° C./min or greater. The cryoprotective composition with the cell may be thawed at a rate of 90° C./min or less, 80° C./min or less, or 70° C./min or less.

According to an embodiment, cell preparation for cryopreservation and wash-free thawing can be executed in a fully automated fashion. For example, cell preparation may be executed using a FLUENT® 780 Workstation (available from Tecan Group Ltd. in Männedorf, Switzerland), an automated incubator at 37° C., 85% humidity, and 5% CO₂, and an automated cell imaging reader such as the CYTATION 1 (available from BioTek Instruments, Inc. in Winooski, VT).

In at least some embodiments, the cells do not need to be washed after thawing. In other words, the cells may be used immediately after thawing without a washing step.

### EXAMPLES

The ability of cryopreservative compositions to preserve cells was tested as follows.

### Example 1

### Cell Line and Maintenance of Culture

Induced pluripotent stem (iPS) cell lines (iPS-DF19-9-11T.H (WiCell) and UMN PCBC16iPSV (or vShiPS 9-1)) were used to develop the cryopreservation method. Cells were cultured in E8 media (TeSR-E8 from STEMCELL Technologies Inc. in Vancouver, Canada and Essential 8 from ThermoFisher Scientific in Waltham, MA) on hESC-qualified MATRIGEL® (from Corning, Inc. in Corning, NY) or recombinant human vitronectin (from PeproTech US in Rocky Hill, NJ) and passaged as aggregates using enzyme-free dissociation reagent ReLeSR (from STEMCELL Technologies) when colonies reached 75-80% confluence.

### Formulation of DMSO-Free Cryopreservation Solution

Basal solution included 5% w/v poloxamer 188 (P188, from Spectrum Chemical in New Brunswick, NJ) in phosphate buffered saline containing Ca²⁺ and Mg²⁺ (PBS++). 2× cryopreservation solution included 60 mM sucrose (from Sigma-Aldrich Corporation in St. Louis, MO), 10% v/v glycerol (from Humco in Austin, TX), 15 mM L-isoleucine (Sigma-Aldrich), 5% w/v P188 and 2×MEM non-essential amino acids (NEAA, Sigma-Aldrich) in PBS++. Mixing basal solution and 2× cryopreservation solution at 1:1 volume ratio resulted in 30 mM sucrose, 5% v/v glycerol, 7.5 mM L-isoleucine, 5% w/v P188, and 1×NEAA in PBS++. The final composition included:

| | |
|---|---|
| Poloxamer 188 | 5% w/v |
| CaCl₂ | 0.90 mM |
| MgCl₂ | 0.49 mM |
| KCl | 2.67 mM |
| KH₂PO₄ | 1.47 mM |
| NaCl | 138 mM |
| Na₂HPO₄ | 8.06 mM |
| Glycine | 0.11 mM |
| Alanine | 0.10 mM |
| Asparagine | 0.10 mM |
| Aspartic acid | 0.10 mM |
| Glutamic acid | 0.10 mM |
| Proline | 0.10 mM |
| Serine | 0.10 mM |

### Cell Preparation for Cryopreservation

Cells were dissociated using ReLeSR™ and harvested as aggregates without centrifugation in the basal solution at cell concentration of ~3×10⁶/ml. Suspension of cell aggregates was loaded into cryovials (Nunc CryoTube, ThermoFisher Scientific) for 0.5 ml/vial. 2× cryopreservation solution was introduced to the cells dropwise for 0.5 ml/vial. Cells were then incubated at room temperature for exactly 30 min before freezing.

### Controlled-Rate Freezing

Cryovials of cells were frozen using a controlled-rate freezer (Kryo 10 Series III from Planer PLC in Middlesex, UK) following the steps listed below using a cooling rate, B, of 1° C./min and a seeding (or ice nucleation) temperature, T_{NUC}, of −4° C.:
1. Starting temperature 20° C.
2. −10° C./min to 0° C.
3. Hold at 0° C. for 10 min to equilibrate temperature inside and outside cryovials
4. −1° C./min to −4° C.
5. Hold at −4° C. for 15 min
6. Induce ice nucleation when cryovial internal temperature reaches −4° C. towards the end of step 5
7. −1° C./min to −60° C.
8. −10° C./min to −100° C.

Frozen cryovials were transferred in a CRYOPOD Carrier (from Biocision, LLC in San Rafael, CA) and stored in liquid nitrogen.

### Wash-Free Thawing

Frozen cryovials were thawed in a 37° C. water bath. The cryovial was submerged below the lid and agitated for 2.5 min.

E8 media, either with or without apoptosis inhibitors, was added to the thawed suspension of cell aggregates using a flow rate of ~1 ml/min. Dilution factor of 2 was used, meaning each cryovial of cells was used to produce 1 well of a 6-well plate of post-thaw cell culture. Diluted cells were seeded onto a freshly coated culture vessel and placed in 5% CO₂ at 37° C. incubator undisturbed for 24 h.

### Experimental Results

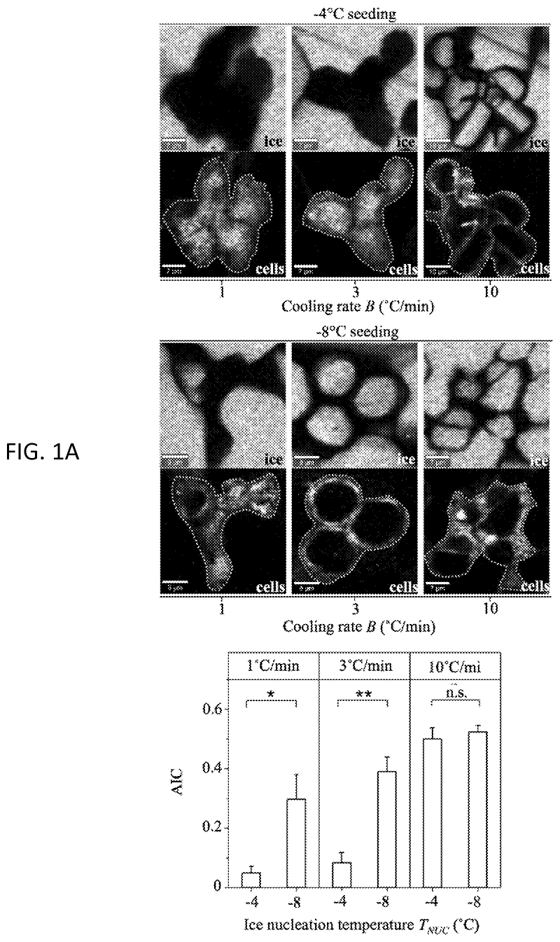

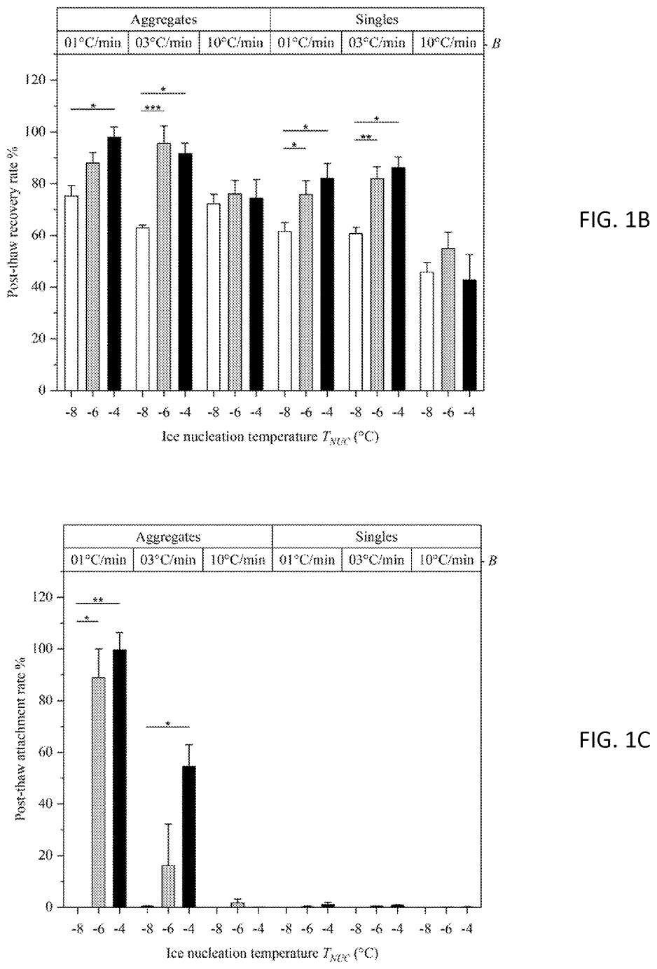

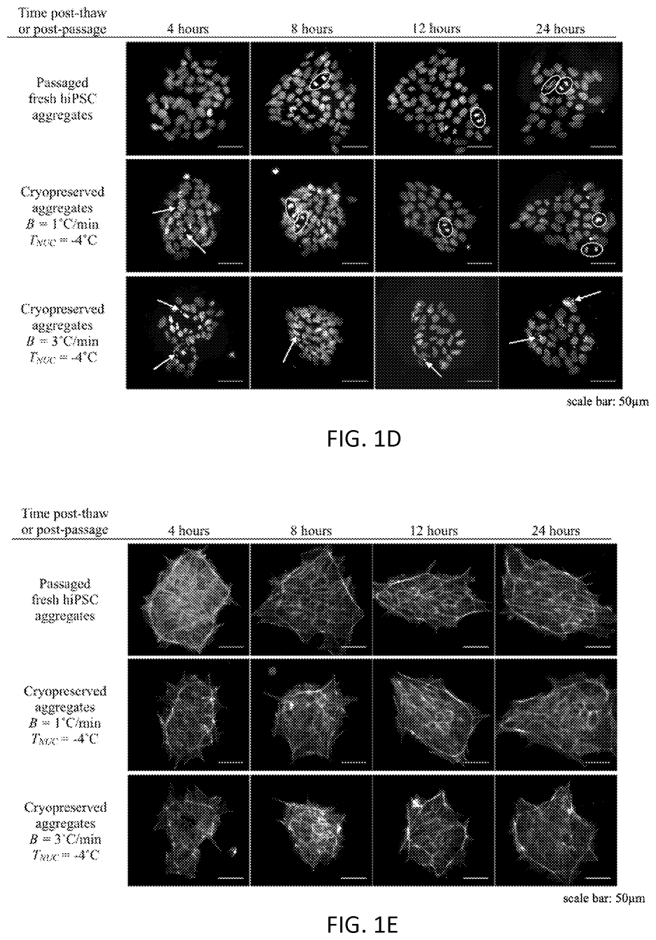

Referring now to FIGS. 1A-1E, in FIG. 1A top panel: Raman heat maps of ice at −50° C. and iPS cell aggregates seeded at −4° C. and frozen with varied cooling rates showed little to no intracellular ice for 1 and 3° C./min but large quantity and size of intracellular ice crystals for 10° C./min. Middle panel: Raman heat maps of ice at −50° C. and cell aggregates seeded at −8° C. and frozen with varied cooling rates showed intracellular ice formation in all aggregates, quantity and size of intracellular ice crystals increasing with cooling rate. Bottom panel: AIC measured for varied cooling rates and ice nucleation temperatures showed statistically significant lower amount of intracellular ice for the higher ice nucleation temperature. n.s.: p≥0.05; * p<0.05; ** p<0.01; *** p<0.001. In FIG. 1B, membrane integrity-based post-thaw recovery with varied cooling rates and ice nucleation temperatures showed statistically significant difference between ice nucleation at −8° C., −6° C., and −4° C., as well as similarity between cryopreserved iPS cell aggregates and single cells. In FIG. 1C, esterase activity-based post-thaw reattachment with varied cooling rates and ice nucleation temperatures showed statistically significant difference between ice nucleation at −8° C., −6° C., and −4° C., that little to no viable culture was resulted from cryopreserved single iPS cells, and high selectivity of this metric to develop freezing conditions for iPS cells. In FIG. 1D, colonies are shown in culture 4, 8, 12, and 24 h post-passage or post-thaw, stained with Hoechst 33342. White arrows point at condensed chromatin, a sign of apoptosis. White circles highlight formed, aligned, or separated sister chromatids, evidence of mitosis. Scale bar: 50 μm. In FIG. 1E, colonies are shown in culture 4, 8, 12, and 24 h post-passage or post-thaw, stained for f-actin. The honeycomb-like pattern is clearly visible around 8 h post-passage and around 12 h post-thaw. Scale bar: 50 μm.

As shown in FIG. 1A, combination of a cooling rate of 1° C./min and ice nucleation temperature of −4° C. inhibited intracellular ice formation (IIF), which protects cell membrane, organelles, and cytoskeleton from ice damage. Lower ice nucleation temperature resulted in statistically significant greater overall amount of IIF, while higher cooling rates did not result in statistically significant difference in IIF but qualitatively larger ice crystals and more disintegrated cell aggregates. As shown in FIG. 1B, the freezing conditions resulted in post-thaw cell recovery of 98.5%. The minimal cell death was consistent with the minimal IIF observed by Raman spectroscopy. Higher cooling rates or lower ice nucleation temperatures resulted in poorer cell recovery. Despite that it yielded similar amount of live cells post-thaw to cryopreserving cell aggregates under varied conditions, cryopreserving singularized iPS cells was not able to produce post-thaw culture without Rho-kinase (“ROCK”) inhibitor no matter the cooling rate or ice nucleation temperature, as seen in FIG. 1C. However, under low cooling rates of 1 or 3° C./min combined with high ice nucleation temperatures of −4 or −6° C., cryopreserving iPS cells as aggregates yielded post-thaw culture successfully without using ROCK inhibitor. And under these conditions, post-thaw reattachment of cell aggregates cryopreserved was comparable to fresh cells, along with early onset of cell proliferation no later than 8 hours (FIG. 1D) and normal honeycomb-like f-actin network formed within 24 hours in its post-thaw culture (FIG. 1E).

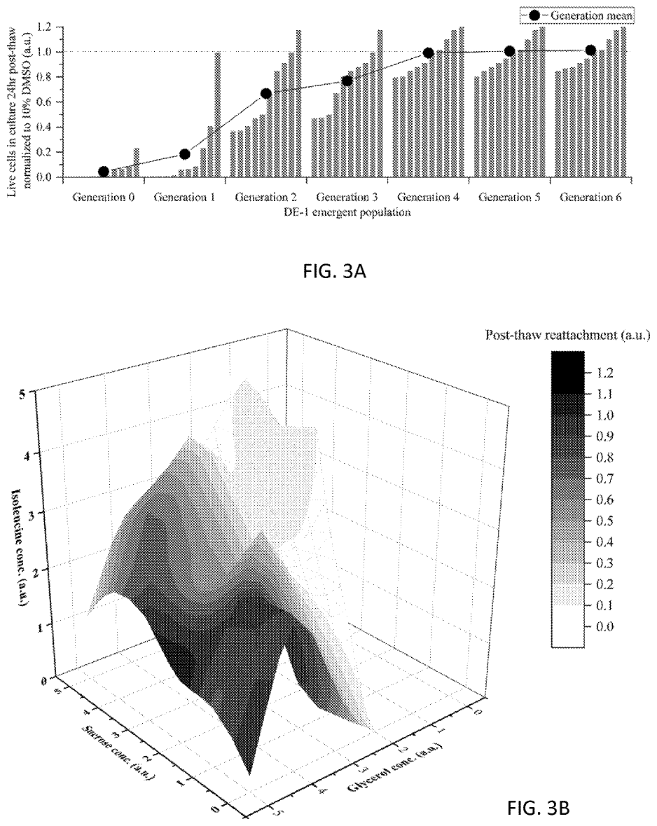

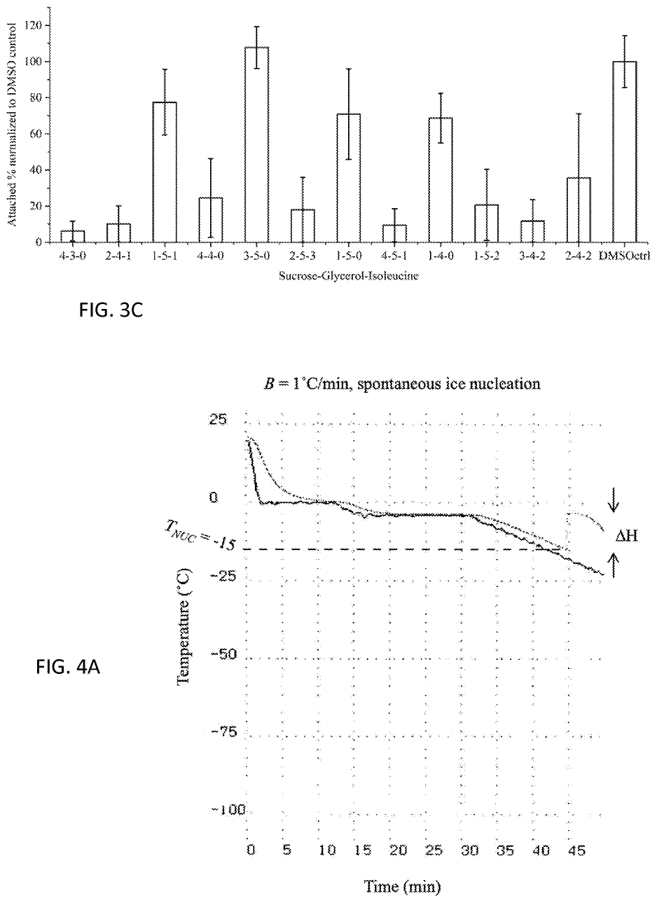

Despite greater amount of cell apoptosis and later onset of proliferation (FIG. 1D), freezing conditions slightly offset from nucleation at −4° C. and cooling rate of 1° C./min (i.e., cooling rate of 3° C./min or ice nucleation of −6° C.) nonetheless demonstrated certain preservation of live iPS cell aggregates (FIG. 1B) with minimal ice damage (FIG. 1A), ability to produce post-thaw culture (FIG. 1C) and recover normal f-actin organization (FIG. 1E), and normal growth and pluripotency to be tested. While manual induction of ice nucleation (FIG. 3A) requires additional technical training, automated induction of ice nucleation (FIG. 3B) is labor-free and achievable by liquid nitrogen-cooled CRF. While liquid nitrogen-free CRF is limited by its working range of cooling rates, unable to perform automated ice nucleation by rapid cooling, it can hold sample temperature slightly below −4° C. to induce ice nucleation theoretically. In some cases, a cooling rate (1 to 3° C./min) may be combined with the nucleation temperature (−4 to −6° C.), as spontaneous ice nucleation can occur as low as −15° C. (FIG. 3C). While spontaneous ice nucleation occurs, cryopreservation is likely to result in significant loss of cell viability and function after thawing and inability to produce viable post-thaw culture. This is a major pain point of freezing containers such as NALGENE® MR. FROSTY™ and a good reason to use CRF for iPS cell cryopreservation.

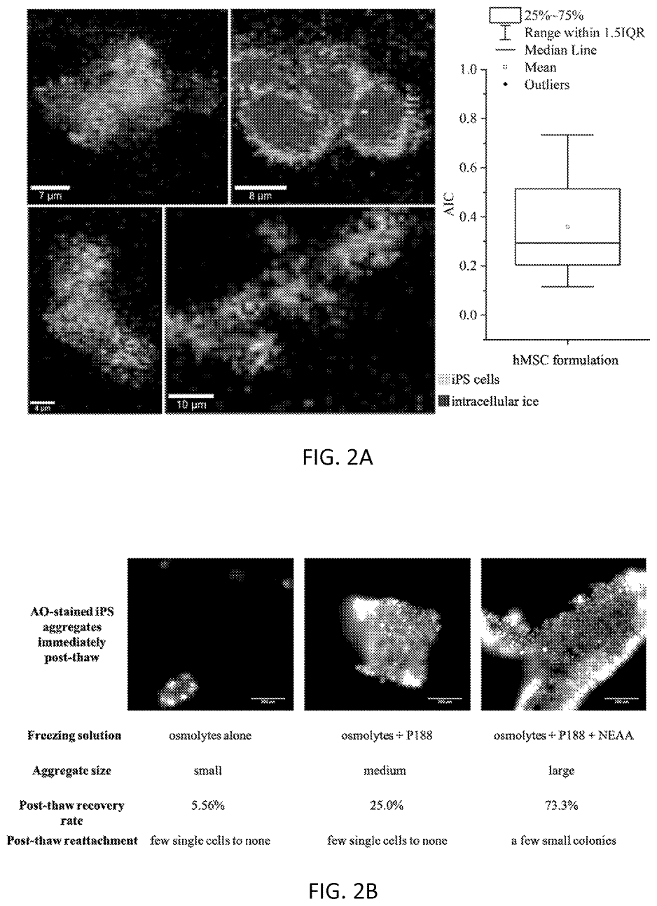

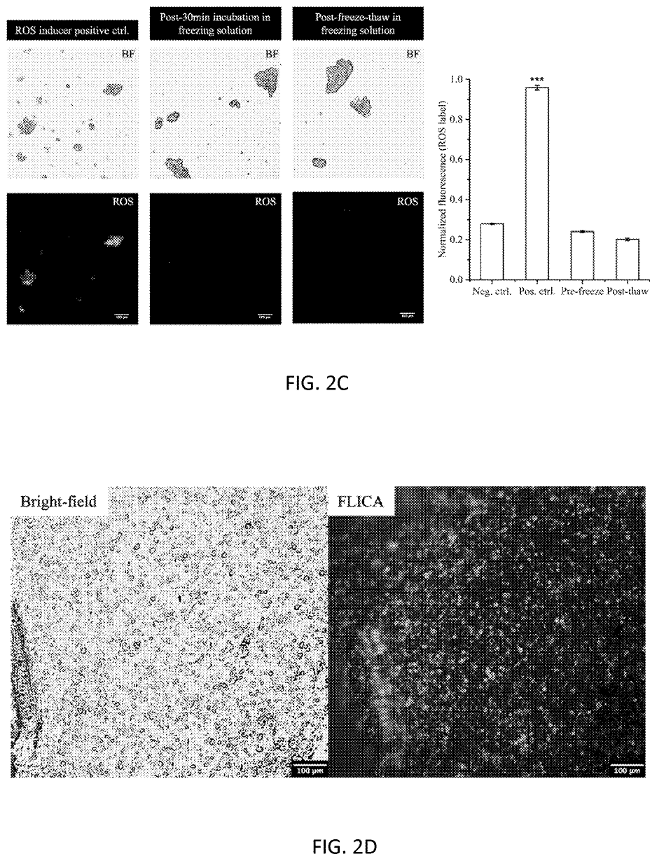

Referring now to FIGS. 2A-2D, in FIG. 2A left: merged Raman heat maps of iPS cell aggregates and ice showed certain ice propagation into cells from the periphery of all 4 sample cell aggregates as well as large ice crystals originated from the cytoplasm of each cell in 1 aggregate (upper right). Right: box chart of AIC showed high dispersion in its statistical distribution indicating highly variable IIF. In FIG. 2B, cell aggregates were cryopreserved using either osmolytes alone, with P188, or with both P188 and NEAA. Qualitative and quantitative analysis of the cryopreserved cells showed positive effects of adding P188 and NEAA on preserving cell-cell contact, membrane integrity and viability. In FIG. 2C, cell aggregates were processed in the cryopreservation solution from (B), which contained osmolytes, P188, NEAA in Normosol R. Bright-field and fluorescent images of ROS label-stained cell aggregates were acquired, after treatment in ROS inducer (1^{st} column), after pre-freeze cell processing (2^{nd} column) and after freeze-thaw (3^{rd} column). Shown was negative detection of oxidative stress in processed cells pre-freeze and post-thaw, and no statistically significant difference was found in comparison to an untreated stained negative control. In FIG. 2D, brightfield and fluorescent images of FAM-FLICA-stained cell pellet showed abundance of apoptotic cell population.

The present composition reduces the occurrence of highly variable outcomes in IIF for iPS cell aggregates (FIG. 2A). This may be attributed at least in part to the presence of poloxamer P188, and NEAA or HBSS buffer. P188 and NEAA may help preserve iPS cell aggregates and produce viable post-thaw culture. Cell aggregates frozen in the hMSC (human mesenchymal stem cell) formulation without P188 or NEAA experienced structural disintegration, nearly complete cell death and did not produce a viable post-thaw culture (FIG. 2B, left). With the addition of P188, integrity of the cell aggregates in cell-cell contact was improved, but without NEAA, majority of cell population was lost during cryopreservation to lysis or apoptosis, and no viable post-thaw culture was produced (FIG. 2B, middle). When P188 and NEAA were both added, structural integrity and viability of the cryopreserved cell aggregates were both improved, as evidence by the large aggregate size and high post-thaw recovery of 73.3%. However, with Normosol R as the buffer, this formulation adapted from hMSCs had very low yield of viable colonies in post-thaw culture (FIG. 2B, right). This low yield was not caused by oxidative stress, as no reactive oxygen species (ROS) or free radicals were detected in the cryopreserved cells (FIG. 2C), but was associated with high level of early apoptosis in the post-thaw cell aggregates, seen in the pellet of cryopreserved cells with mostly FAM-FLICA-positive population (FIG. 2D).

When Normosol R was replaced with PBS++ as the buffer, post-thaw reattachment rose slightly to 27%, but apoptosis was still outstanding in the cryopreserved cells. Screening of various apoptotic pathways suggested that Rho kinase-mediated intrinsic apoptosis, Fas ligand and TRAILR2-mediated extrinsic apoptosis began in the cells upon pre-freeze incubation in the cryopreservation solution, and the extrinsic apoptosis was advanced further in response to freeze-thaw (TABLE 1, 2).

TABLE 1
Post-thaw reattachment rate of cell aggregates buffered in PBS++^{a}

| | Rho | Pan | Fas | TRAIL | TRAIL | TRAIL |
|---|---|---|---|---|---|---|
| Target of inhibition | kinase | caspase | ligand | R1 | R2 | R4 |
| Inhibitor added pre-freeze | 109% | 15% | 45% | 2% | 40% | 2% |
| Inhibitor added post-thaw | 11% | 30% | 62% | 1% | 51% | 2% |

^{a}100%: same as DMSO control; without inhibitor = 27%

TABLE 2
Change in post-thaw cell reattachment rate in PBS++ upon apoptosis inhibition

| | Rho | Pan | Fas | TRAIL | TRAIL | TRAIL |
|---|---|---|---|---|---|---|
| Target of inhibition | kinase | caspase | ligand | R1 | R2 | R4 |
| Inhibitor added pre-freeze | +301% | −44% | +66% | −92% | +48% | −94% |
| Inhibitor added post-thaw | −58% | +12% | +127% | −96% | +89% | −92% |

^{a}0%: same as without inhibitor,
+positive effect of adding inhibitor,
−negative effect

When PBS++ was replaced with HBSS as the buffer, post-thaw reattachment improved drastically to 96% (TABLE 3). Moreover, both extrinsic and intrinsic apoptosis were diminished (TABLE 4). Contrary to common practice in cryopreservation of iPS cells, which uses Y-27632 and other apoptosis inhibitors to rescue poorly preserved and highly apoptotic cells, all apoptosis inhibitors showed a significant negative effect on the cryopreserved cells and reduced cell proliferation in the post-thaw culture (TABLE 5). The cryopreservation outcome of the composition exceeded that of DMSO-based formulation with improved cell proliferation and became independent from apoptosis inhibitors.

TABLE 3
Post-thaw reattachment rate of cell aggregates buffered in HBSS^{a}

| | Rho | Pan | Fas | TRAIL | TRAIL | TRAIL |
|---|---|---|---|---|---|---|
| Target of inhibition | kinase | caspase | ligand | R1 | R2 | R4 |
| Inhibitor added pre-freeze | 104% | | 76% | | 95% | |
| Inhibitor added post-thaw | | 100% | 106% | | 42% | |

^{a}100%: same as DMSO control; without inhibitor = 96%

TABLE 4
Change in post-thaw cell reattachment rate in HBSS upon apoptosis inhibition^{a}

| | Rho | Pan | Fas | TRAIL | TRAIL | TRAIL |
|---|---|---|---|---|---|---|
| Target of inhibition | kinase | caspase | ligand | R1 | R2 | R4 |
| Inhibitor added pre-freeze | +8% | | −21% | | −1% | |
| Inhibitor added post-thaw | | −3% | −10% | | −56% | |

^{a}0%: same as without inhibitor,
+positive effect of adding inhibitor,
−negative effect

TABLE 5
Cell proliferation of culture cryopreserved in HBSS from 24 to 48 h post-thaw^{a}

| Controls | | Inhibitors added pre-freeze | | | Inhibitors added post-thaw | | |
|---|---|---|---|---|---|---|---|
| Basal | DMSO | Rho | Fas | TRAIL | Pan | Fas | TRAIL |
| DMSO-free | control | kinase | ligand | R2 | caspase | ligand | R2 |
| 3.596 | 0.536 | 0.967 | 1.713 | 2.396 | 3.312 | 1.749 | 0.836 |

^{a}1: no net change in total live cell count in culture

The final formulation included albumin, sucrose, glycerol, isoleucine, P188, NEAA and HBSS. The composition of HBSS is shown in TABLE 6 below.

TABLE 6
Compositions of Hank's Balanced Salts Solution (HBSS)

| Component | Molarity (mM) |
|---|---|
| Calcium Chloride (CaCl₂, anhydrous) | 1.26 |
| Magnesium Chloride (MgCl₂—6H₂O) | 0.49 |
| Magnesium Sulfate (MgSO₄—7H₂O) | 0.41 |
| Potassium Chloride (KCl) | 5.33 |
| Potassium Phosphate monobasic (KH₂PO₄) | 0.44 |
| Sodium Bicarbonate (NaHCO₃) | 4.17 |
| Sodium Chloride (NaCl) | 137.93 |
| Sodium Phosphate dibasic (Na₂HPO₄, | 0.34 |
| anhydrous) | |
| D-Glucose (Dextrose) | 5.56 |

It was also found in preliminary screening of osmolytes that iPS cells had similar dosage response across various disaccharides and better response to cell-permeant than impermeant sugar alcohols (TABLE 7). Alternative disaccharide molecules (e.g., trehalose, maltose, lactose), sugar alcohol (e.g., ethylene glycol) and amino acid (e.g., creatine) may be used to replace sucrose, glycerol and isoleucine and yield comparable outcomes of iPS cell cryopreservation.

TABLE 7
Screening of osmolytes for wash-free cryopreservation

| | | Molar | | | | |
|---|---|---|---|---|---|---|
| | | mass | Conditions and observation | | | |
| Molecule | Chemical structure | (g/mol) | 300 mM, 30 min | 150 mM, 30 min | 75 mM, 30 min | 75 mM, 1 h |
| sucrose | 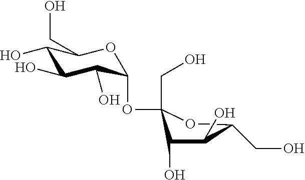 | 342.30 | no colonies, all floaters | no colonies, all floaters | many colonies, normal morphology | fewer colonies, more floaters |
| trehalose | 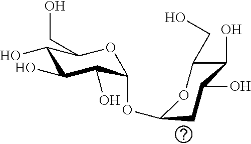 | 342.30 | no colonies, all floaters | no colonies, all floaters | many colonies, normal morphology | fewer colonies, more floaters |
| maltose | 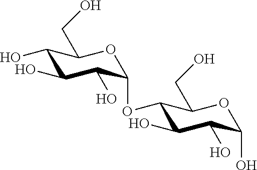 | 342.30 | no colonies, all floaters | no colonies, all floaters | many colonies, normal morphology | fewer colonies, more floaters |
| lactose | 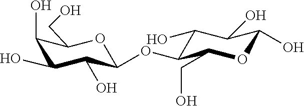 | 342.30 | no colonies, all floaters | no colonies, all floaters | many colonies, normal morphology | fewer colonies, more floaters |
| ethylene glycol | 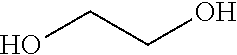 | 62.07 | many colonies, normal morphology | — | — | — |
| glycerol | 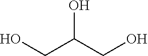 | 92.09 | many colonies, normal morphology | — | — | — |
| D- sorbitol | 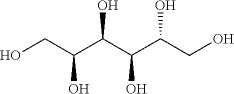 [SMILES: OC[C@@H](O)[C@@H](O)[C@H](O)[C@@H](O)CO] | 182.17 | very few colonies, many floaters | — | — | — |
| D- mannitol | 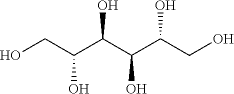 [SMILES: OC[C@@H](O)[C@@H](O)[C@H](O)[C@H](O)CO] | 182.17 | very few colonies, many floaters | — | — | — |
| xylitol | 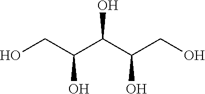 | 152.15 | very few colonies, many floaters | — | — | — |
| myo- inositol | 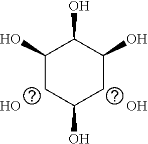 | 180.16 | very few colonies, many floaters | — | — | — |
| adonitol | 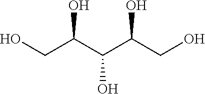 | 152.15 | very few colonies, many floaters | — | — | — |

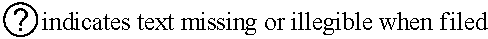

### Example 2

### Cell Line and Maintenance of Culture

Induced pluripotent stem (iPS) cell line UMN PCBC16iPSV (or vShiPS 9-1) was used to develop the cryopreservation method. Cells were cultured in TeSR-E8 media (from STEMCELL Technologies Inc. in Vancouver, Canada) on recombinant human vitronectin (from PeproTech US in Rocky Hill, NJ) and passaged as aggregates using 0.02% ethylenediaminetetraacetic acid solution (from Sigma-Aldrich Corporation in St. Louis, MO) at a split ratio of 1:8 every 4 days, when colony confluence reached 55-65%.

### Formulation of DMSO-Free Cryopreservation Solution

Basal solution included 5% w/v poloxamer 188 (P188, from Spectrum Chemical in New Brunswick, NJ) in Hank's Balanced Salt Solution with Ca²⁺, Mg²⁺, and glucose (HBSS, from Lonza in Basel, Switzerland). 2× cryopreservation solution included 180 mM sucrose (from Sigma-Aldrich Corporation in St. Louis, MO), 10% v/v glycerol (from Humco in Austin, TX), 5% w/v P188, and 2×MEM non-essential amino acids (NEAA, Sigma-Aldrich) in HBSS. Mixing basal solution and 2× cryopreservation solution at 1:1 volume ratio resulted in 90 mM sucrose, 5% v/v glycerol, 5% w/v P188, and 1×NEAA in HBSS. The final composition included:

| Component | Concentration/molarity |
|---|---|
| Sucrose | 90 mM |
| Dextrose | 5.6 mM |
| Glycerol | 684 mM |
| Poloxamer 188 | 5% w/v |
| CaCl₂ | 1.25 mM |
| MgCl₂ | 0.49 mM |
| MgSO₄ | 0.41 mM |
| KCl | 5.3 mM |
| KH₂PO₄ | 0.44 mM |
| NaHCO₃ | 4.2 mM |
| NaCl | 138 mM |
| Na₂HPO₄ | 0.34 mM |
| Glycine | 0.11 mM |
| Alanine | 0.10 mM |
| Asparagine | 0.10 mM |
| Aspartic acid | 0.10 mM |
| Glutamic acid | 0.10 mM |
| Proline | 0.10 mM |
| Serine | 0.10 mM |

### Cell Preparation for Cryopreservation

Cells were dissociated using ReLeSR™ and harvested as aggregates without centrifugation in the basal solution at cell concentration of ~3×10⁶/ml. Suspension of cell aggregates was loaded into cryovials (Nunc CryoTube, ThermoFisher Scientific) for 0.5 ml/vial. 2× cryopreservation solution was introduced to the cells dropwise for 0.5 ml/vial. Cells were then incubated at room temperature for exactly 30 min before freezing.

### Controlled-Rate Freezing

Cryovials of cells were frozen using a controlled-rate freezer (Kryo 10 Series III from Planer PLC in Middlesex, UK) following the steps listed below using a cooling rate, B, of 1° C./min and a seeding (or ice nucleation) temperature, T_{NUC}, of −4° C.:
1. Starting temperature 20° C.
2. −10° C./min to 0° C.
3. Hold at 0° C. for 10 min to equilibrate temperature inside and outside cryovials
4. −1° C./min to −4° C.
5. Hold at −4° C. for 15 min
6. Induce ice nucleation when cryovial internal temperature reaches −4° C. towards the end of step 5
7. −1° C./min to −60° C.
8. −10° C./min to −100° C.

Frozen cryovials were transferred in a CRYOPOD Carrier (from Biocision, LLC in San Rafael, CA) and stored in liquid nitrogen.

### Wash-Free Thawing

Frozen cryovials were thawed in either a 37° C. water bath or a THAWSTAR® Automated Thawing System (Biocision). If using 37° C. water bath, cryovial was submerged below the lid and agitated for 2.5 min. If using ThawSTAR, cryovial was agitated for 20 s after the device indicated that the sample had thawed.

TeSR-E8 media, without apoptosis inhibitors or other additives, was added to the thawed suspension of cell aggregates using a flow rate of ~1 ml/min. Dilution factors of 2 was used, meaning each cryovial of cells was used to produce 1 well of a 6-well plate of post-thaw cell culture. Diluted cells were seeded onto freshly coated culture vessel and placed in 5% CO₂ at 37° C. incubator undisturbed for 24 h.

### Experimental Results

Referring now to FIGS. 3A-3C, in FIG. 3A, differential evolutionary algorithm-driven development of DMSO-free cryopreservation solution converged as the emergent population was repeated at generation 6. Post-thaw cell reattachment as the metric was normalized to a DMSO control. The DMSO control formulation contained 10% v/v DMSO and 5% w/v P188 in HBSS. FIG. 3B shows a 4D plot of post-thaw cell reattachment with varied concentration of sucrose, glycerol, and isoleucine. The topography of this parameter space in terms of post-thaw cell reattachment was non-linear, not unimodal, but complex with closely-spaced contour lines within the region containing the optimum, but smooth in the distance from the optimum. In FIG. 3C, post-thaw reattachment rate of the best 12 formulations from the algorithm-driven development was validated with four independent biological replicates in comparison with the 10% DMSO control. High variability in post-thaw cell reattachment was seen for multiple formulations (e.g. formulation #2-4-2), whereas low variability as well as high post-thaw cell reattachment was seen for formulation #3-5-0.

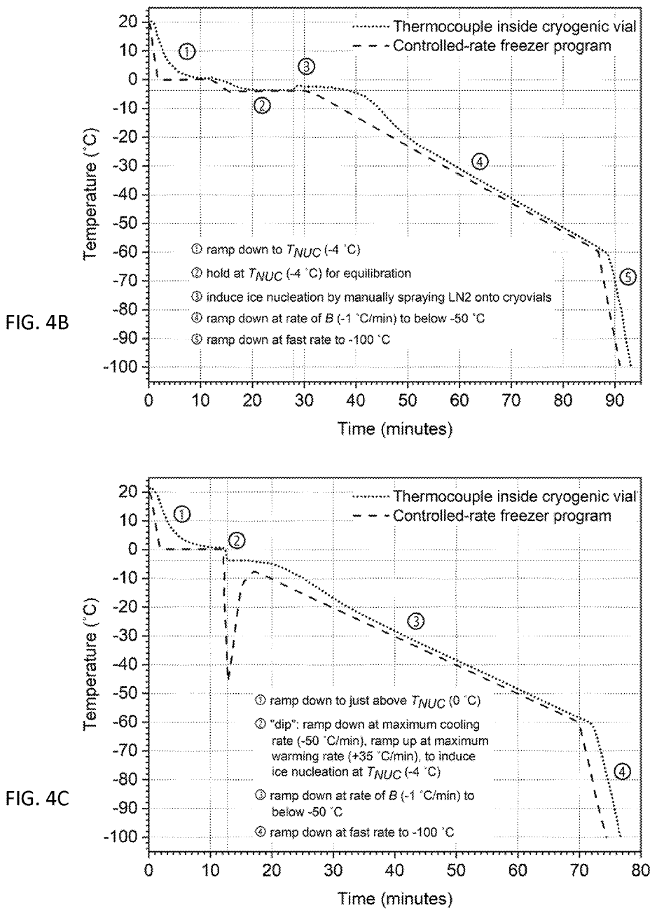

FIGS. 4A-4C show cooling profiles used in the Examples recorded of varied methods of inducing ice nucleation under a constant cooling rate of 1° C./min, labeled with ice nucleation temperature, T_{NUC}, and latent heat, ΔH. In the figures, dashed line: programmed CRF chamber temperature; solid line: recorded CRF chamber temperature (or cryovial external temperature); dotted line: recorded sample temperature (or cryovial internal temperature). In FIG. 4A, spontaneous ice nucleation occurred at −15° C. but not during 15 min hold at −4° C., with massive release of latent heat indicating great amount of undesirable ice formation. In FIG. 4B, manually opening CRF and spraying liquid nitrogen rapidly onto cryovials successfully induced ice nucleation as expected at −4° C., with minimal release of latent heat indicating well inhibited ice formation. In FIG. 4C, automated rapid cooling of CRF chamber successfully induced ice nucleation slightly below the expected −4° C., with minimal release of latent heat indicating well inhibited ice formation.

### Example 3

### Cell Line and Maintenance of Culture

Induced pluripotent stem (iPS) cell line UMN PCBC16iPSV (or vShiPS 9-1) was used to develop the cryopreservation method, and additional in-house and commercialized iPS cell lines (UMN AMD3-6B4, ACS 1024 (ATCC), and hiPSC-CCND2OE) were used to validate the method. Cells were cultured in TeSR-E8 media (from STEMCELL Technologies Inc. in Vancouver, Canada) on hESC-qualified MATRIGEL® (from Corning, Inc. in Corning, NY) or recombinant human vitronectin (from PeproTech US in Rocky Hill, NJ) and passaged as aggregates using enzyme-free dissociation reagent ReLeSR (STEMCELL Technologies) at a split ratio of 1:8 every 4 days, when colony confluence reached 65-75%.

### Formulation of DMSO-Free Cryopreservation Solution

Basal solution included 5% w/v poloxamer 188 (P188, from Spectrum Chemical in New Brunswick, NJ) in Hank's Balanced Salt Solution with Ca²⁺, Mg²⁺, and glucose (HBSS, from Lonza in Basel, Switzerland). 2× cryopreservation solution included 120 mM sucrose (from Sigma-Aldrich Corporation in St. Louis, MO), 10% v/v glycerol (from Humco in Austin, TX), 15 mM L-isoleucine (Sigma-Aldrich), 5% w/v P188, 4% albumin (ALBUTEIN®, Grifols S.A. in Barcelona, Spain), and 2×MEM non-essential amino acids (NEAA, Sigma-Aldrich) in HBSS. Mixing basal solution and 2× cryopreservation solution at 1:1 volume ratio resulted in 60 mM sucrose, 5% v/v glycerol, 7.5 mM L-isoleucine, 5% w/v P188, 2% albumin, and 1×NEAA in HBSS. The final composition included:

| Component | Concentration/molarity |
|---|---|
| Sucrose | 60 mM |
| Dextrose | 5.6 mM |
| Glycerol | 684 mM |
| L-isoleucine | 7.5 mM |
| Poloxamer 188 | 5% w/v |
| Albumin | 2% w/v |
| CaCl₂ | 1.25 mM |
| MgCl₂ | 0.49 mM |
| MgSO₄ | 0.41 mM |
| KCl | 5.3 mM |
| KH₂PO₄ | 0.44 mM |
| NaHCO₃ | 4.2 mM |
| NaCl | 138 mM |
| Na₂HPO₄ | 0.34 mM |
| Glycine | 0.11 mM |
| Alanine | 0.10 mM |
| Asparagine | 0.10 mM |
| Aspartic acid | 0.10 mM |
| Glutamic acid | 0.10 mM |
| Proline | 0.10 mM |
| Serine | 0.10 mM |

### Cell Preparation for Cryopreservation

Cells were dissociated using ReLeSR™ and harvested as aggregates without centrifugation in the basal solution at cell concentration of ~3×10⁶/ml. Suspension of cell aggregates was loaded into cryovials (Nunc CryoTube, ThermoFisher Scientific) for 0.5 ml/vial. 2× cryopreservation solution was introduced to the cells dropwise for 0.5 ml/vial. Cells were then incubated at room temperature for 1 hour before freezing.

### Controlled-Rate Freezing

Cryovials of cells were frozen using a controlled-rate freezer (Kryo 10 Series III from Planer PLC in Middlesex, UK) following the steps listed below using a cooling rate, B, of 1° C./min and a seeding (or ice nucleation) temperature, T_{NUC}, of −4° C. or −12° C.:
1. Starting temperature 20° C.
2. −10° C./min to 0° C.
3. Hold at 0° C. for 10 min to equilibrate temperature inside and outside cryovials
4. −1° C./min to T_{NUC}
5. Hold at T_{NUC} for 15 min
6. Induce ice nucleation when cryovial internal temperature reaches T_{NUC} towards the end of step 5
7. −B° C./min to −60° C.
8. −10° C./min to −100° C.

Frozen cryovials were transferred in a CRYOPOD Carrier (from Biocision, LLC in San Rafael, CA) and stored in liquid nitrogen.

### Passive Freezing

Alternative to controlled-rate freezing, passive freezing was performed using an insulated freezing container CoolCell (BioCision). Cryovials of cells were placed inside the CoolCell at room temperature and frozen inside a −80° C. mechanical freezer over a period of 4 hours. Sample temperature was logged using a DI-245 USB Thermocouple Data Acquisition system (from DATAQ Instruments in Akron, OH). The thermocouple was inserted via a holed, fitted cap into a “dummy” vial containing the same volume of cells and cryopreservation solution as the experimental samples. After freezing, the cryovials were transferred in the CRYOPOD Carrier and stored in liquid nitrogen.

### Wash-Free Thawing

Frozen cryovials were thawed in either a 37° C. water bath or a THAWSTAR® Automated Thawing System (Biocision). If using 37° C. water bath, cryovial was submerged below the lid and agitated for 2.5 min. If using ThawSTAR, cryovial was agitated for 20 s after the device indicated that the sample had thawed.

Thawed suspension of cell aggregates was transferred, without centrifugation or other additives, into E8 media using a flow rate of ~1 ml/min. Dilution factors between 2 and 15 were used, meaning each cryovial of cells was used to produce between 1 well of a 6-well plate and 1 T75 flask of post-thaw cell culture. Diluted cells were seeded onto freshly coated culture vessel and placed in 5% CO₂ at 37° C. incubator undisturbed for 24 h. The post-thaw culture was either passaged to maintain cell culture or directly used for downstream workflow such as directed differentiation. FIG. 5 shows an illustration of the entire cryopreservation workflow described above.

### Experimental Results

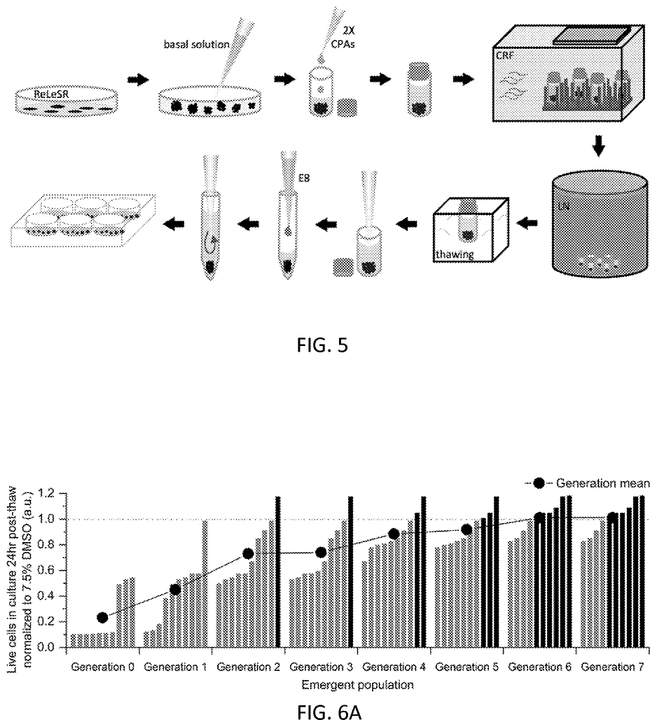

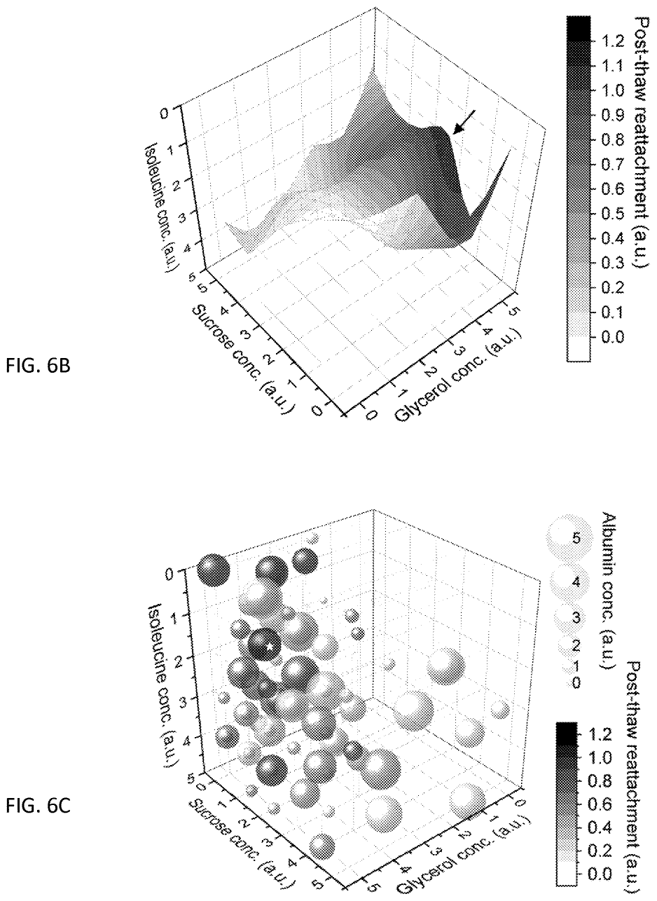

Referring now to FIGS. 6A-6C, in FIG. 6A, differential evolutionary algorithm-driven development of DMSO-free cryopreservation solution converged as the emergent population was repeated at generation 7, or after 8 sets of 10-sample experiments, in a parameter space of 4 variable parameters, sucrose, glycerol, isoleucine and albumin that was each discretized into 5 intervals. Post-thaw cell reattachment as the metric was normalized to a DMSO control. The DMSO control formulation contained 7.5% v/v DMSO and 5% w/v P188 in HBSS. A range of DMSO-free formulations exceeded the DMSO control in post-thaw cell reattachment. These formulations contained 20-60 mM sucrose, 4-5% v/v glycerol, 0-22.5 mM isoleucine, 0-2% albumin, 5% w/v P188, 1×NEAA and HBSS. FIG. 6B shows a 4D landscape of the parameter space that the DE algorithm navigated through during development, shown in 4 of 5 dimensions. The topology of this parameter space was non-linear and not unimodal, but complex with closely spaced contour lines within the region containing the optimum as pointed out by the arrow. FIG. 6C shows a 5D bubble chart of all formulations tested by the DE algorithm, shown in all 5 dimensions. Optimum (star) located at level-2 sucrose, level-5 glycerol, level-1 isoleucine, level-4 albumin.

### Effective Cryopreservation of iPS Cell Aggregates

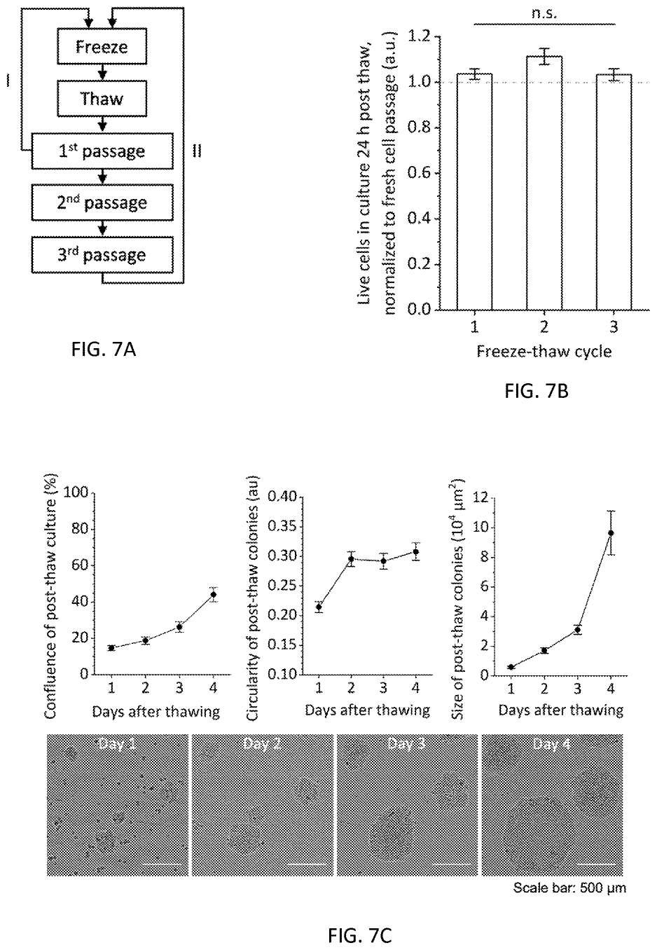

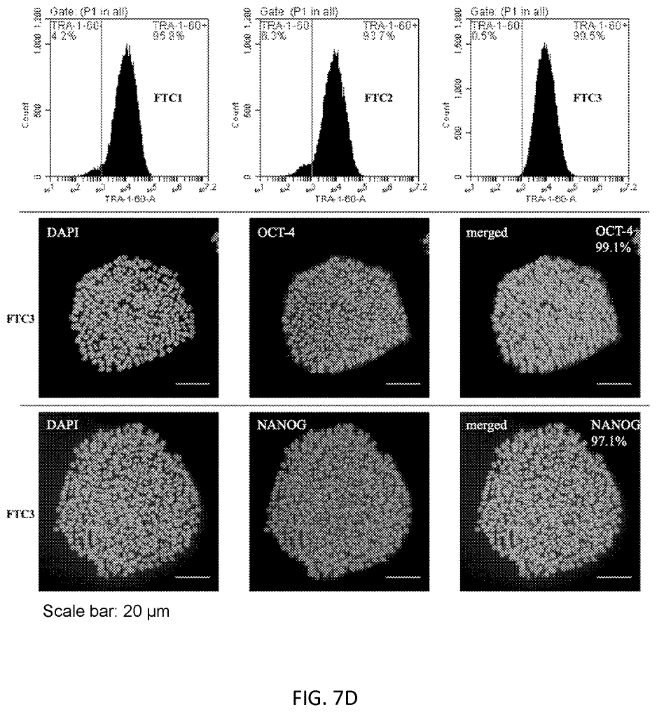

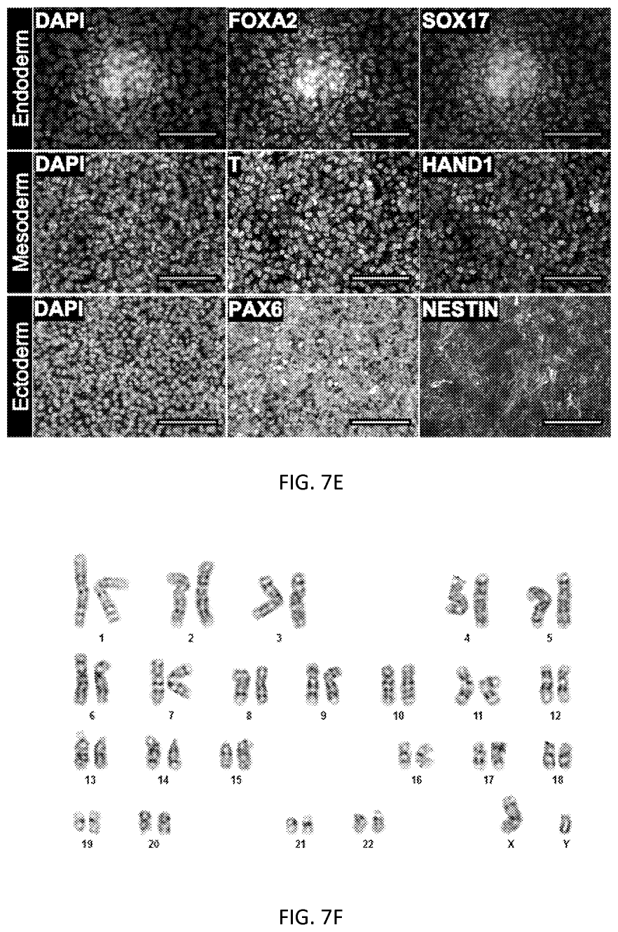

Referring now to FIG. 7A, the cryopreservation method was stress-tested via three freeze-thaw cycles. Type-I cycle of freezing, thawing and one passage of post-thaw culture was used to amplify any phenotypic instability that could result from cryopreservation, and FIG. 7B-7E were resulted from type-I cycle. Type-II cycle of freezing, thawing and three passages of post-thaw culture was used to amplify any chromosomal instability that could result from cryopreservation, and FIG. 7F was resulted from type-II cycle.

In FIG. 7B, the cryopreserved cell aggregates showed strong capability to re-establish culture, evidenced by its post-thaw reattachment, comparable to fresh cells. At 24 hours after thawing, the amount of live cells resulted in culture from the DMSO-free cryopreservation had no statistically significant difference from that by passaging fresh cells. This was consistent throughout the 3 wash-free freeze-thaw cycles, where no statistically significant difference in post-thaw attachment was found after any round of cryopreservation. In FIG. 7C, post-thaw culture of the cryopreserved cells showed normal proliferation, colony growth and compaction over 4 days after the third round of thawing as evident by the increase in confluence, recovery of colony circularity, and increase in colony size shown by both the growth curves quantified by automated imaging using a Cytation 1 cell imaging multi-mode reader (BioTek).

In FIG. 7D, the cryopreserved cells also showed normal pluripotency in the freeze-thaw stress test. Expression of surface marker TRA-1-60 was high throughout the 3 rounds of cryopreservation. 95.8% and 93.7% TRA-1-60-positive population were measured immediately post-thaw after the first two rounds respectively, which indicated that the DMSO-free cryopreservation method caused little physico-mechanical damage to the podocalyxin membrane protein. 99.5% TRA-1-60-positive population was measured of 75-80% confluent culture on day 6 after the third round of thawing, slightly higher than the previous two measurements, which indicated not only consistently preserved podocalyxin but effective recovery towards 100% pluripotency of the post-thaw culture under the current protocol. In addition, expression of transcription factors OCT-4 and NANOG were both high in the cryopreserved cells. 99.1% OCT-4-positive and 97.1% NANOG-positive population were measured of the post-thaw culture at passage confluence after the third round of cryopreservation. Along with the TRA-1-60 expression, this represented a highly pluripotent iPS cell culture with little to no spontaneous differentiation. Upon a fourth round of thawing, cryopreserved cells were differentiated into all three germ layers, with positive co-expression of endodermal markers, FOXA2 and SOX17, mesodermal markers, NCAM and T, and ectodermal markers, PAX6 and Nestin, as shown by the fluorescent images in FIG. 7E.

In FIG. 7F, the third passage after the third type-II freeze-thaw cycle, cytogenetic findings of the cryopreserved cells represented a normal male karyotype. No clonal numerical or structural chromosomal abnormality was found among the 16 metaphase cells available for analysis. All findings above demonstrated a highly desirable, effective DMSO-free cryopreservation method.

All findings above demonstrated a highly desirable, effective DMSO-free cryopreservation method.

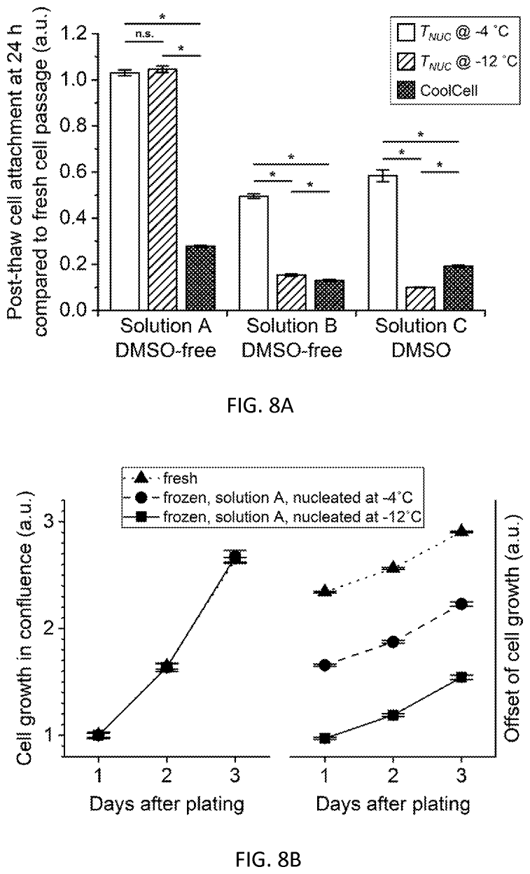

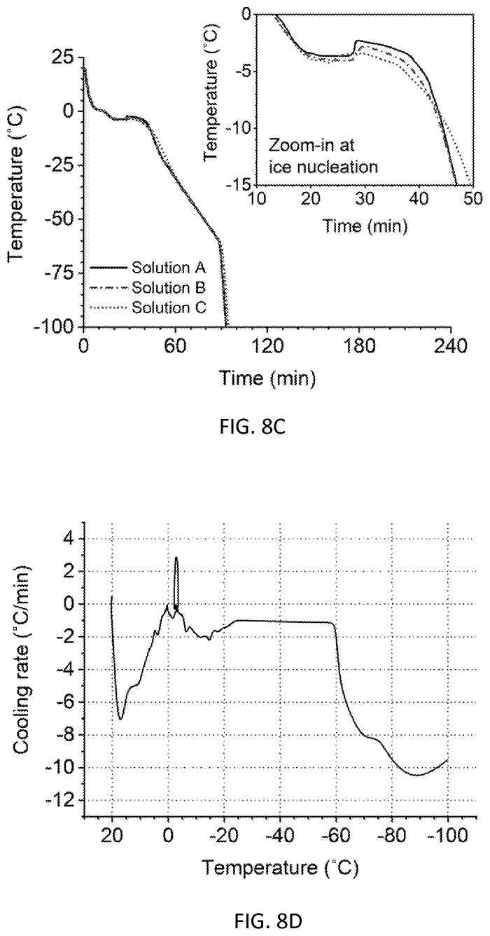

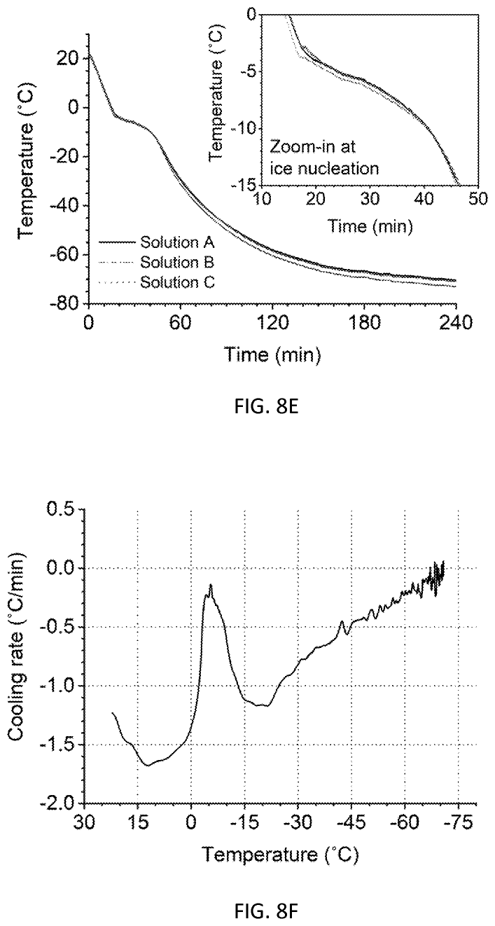

The use of the composition with P188, sucrose, glycerol, isoleucine and albumin results in multicellular aggregates exhibiting far less sensitivity to undercooling. FIG. 8A shows that post-thaw cell attachment was not compromised even under greater extent of undercooling as long as the DMSO-free composition described in the formulation of this freezing method was used, whereas it dropped significantly when the composition slightly deviated from the DMSO-free composition or when DMSO was used. FIG. 8B shows that whether ice nucleation occurred at −4° C. or −12° C., the growth of cryopreserved iPS cells in post-thaw culture was comparable to that of fresh iPS cells after standard passaging.

FIG. 8C shows sample internal temperature recorded over the course of controlled-rate freezing with ice nucleation induced manually at −4° C. FIG. 8D shows the first derivative of FIG. 8C plotted against the corresponding sample internal temperature. FIG. 8E shows sample internal temperature recorded over the course of passive freezing in CoolCell with spontaneous ice nucleation. FIG. 8F shows the first derivative of FIG. 8E plotted against the corresponding sample internal temperature. Regardless of cryopreservation solutions, the use of controlled-rate freezing significantly improved the post-thaw cell survival by mitigating cell sensitivity to undercooling and cooling rate, whereas the use of passive freezing involved lower cooling rates over an extensive period of time and results in significantly cell loss due to cell sensitivity to cooling rate.

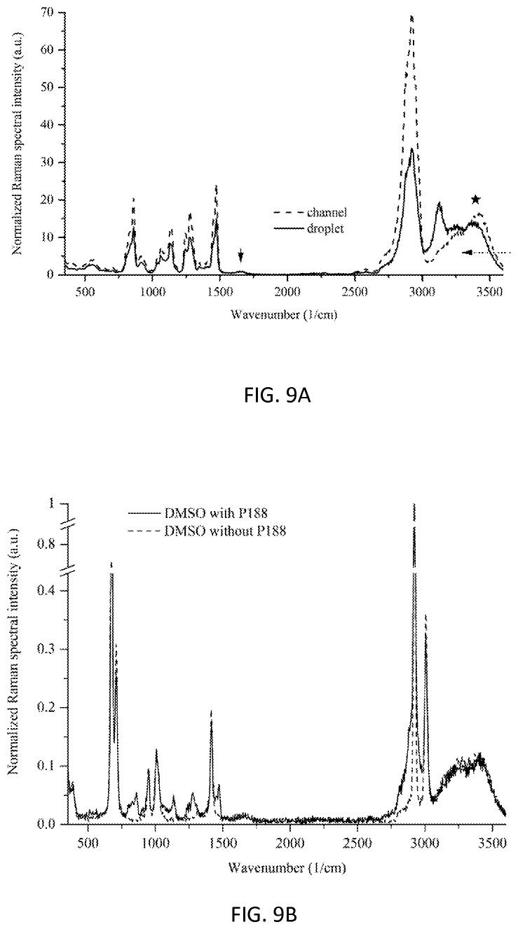

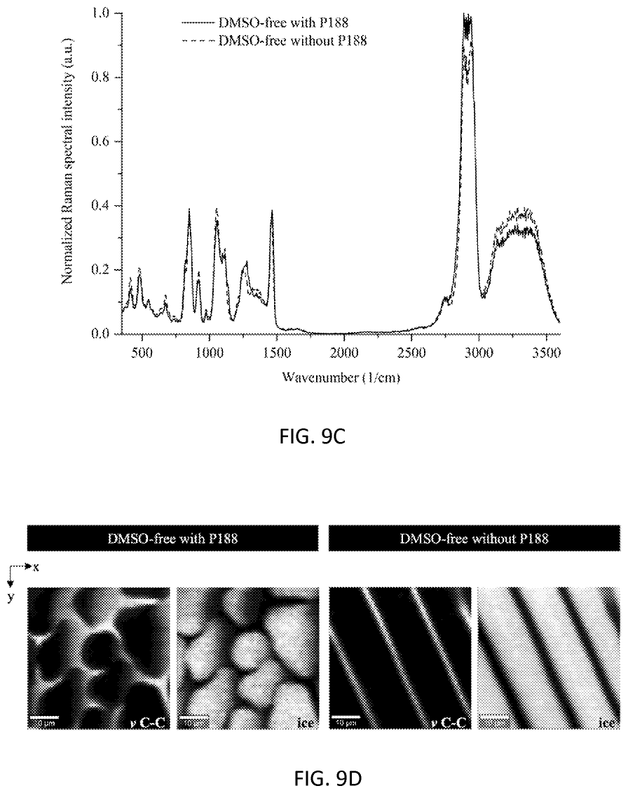

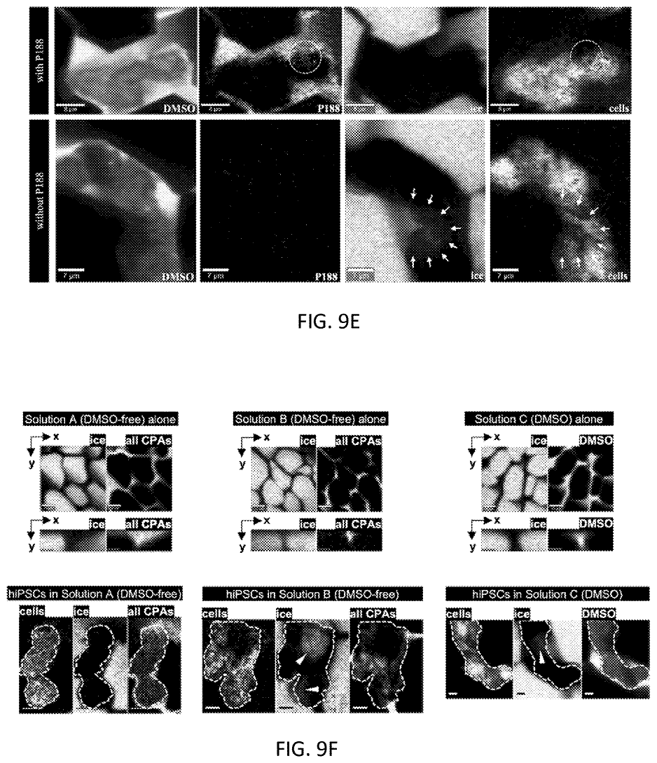

The mechanisms of action of P188 as a cryoprotective agent were attributed to its inhibition of ice formation and its synergistic interactions with the other osmolytes in the composition. FIG. 9A shows Raman spectra acquired in the channel of nonfrozen P188 aqueous solution between ice crystals versus droplet of P188 micelles embedded in ice at −50° C. Red shift of broad OH stretching peak (dashed arrow) and down shift of hydrohalite peak (star) indicated that P188 micelles strengthened hydrogen bond network of water lattice and inhibited hydrohalite formation. Micelles were absent when P188 was mixed with glycerol. FIG. 9B shows Raman spectra of nonfrozen DMSO solution with versus without P188 between ice crystals at −50° C. Difference between the two spectra could be accounted for by superposition of P188's signal onto DMSO. No red shift of broad OH stretching peak upon the addition of P188 indicated no effect on hydrogen bond network. FIG. 9C shows Raman spectra of nonfrozen DMSO-free solution with versus without P188 between ice crystals at −50° C. Difference between the two spectra could be accounted for by superposition of P188's signal onto rest of the DMSO-free solution. No red shift of broad OH stretching peak upon the addition of P188 indicated no effect on hydrogen bond network. Summarizing FIG. 9A-9C, there is no evidence suggesting that P188 in the method of the present disclosure acts to strengthen hydrogen bonding.

In FIG. 9D, Raman heat maps of ice crystals that formed in DMSO-free solution showed distinct difference in ice morphology in samples with versus without P188 at −50° C. in lateral and axial directions. In FIG. 9E, iPS cell aggregates were frozen in DMSO solution with versus without P188 to −50° C. Raman heat maps showed that location of intracellular P188 coincided with that of mild cellular content loss (dashed circles), which demonstrated P188's function as a sealant, and that P188 prevented ice from propagating into the cells and disintegrating the cell aggregate (arrows). In FIG. 9F, Raman heat maps of different DMSO-free and DMSO-based solutions at −50° C. showed that a unique ice morphology that was observed only when P188 was used along with a composition of sucrose, glycerol, isoleucine and albumin. The propagating front of each ice crystal was softened increasing the amount of nonfrozen space between adjacent ice crystals. TABLE 8 shows the effect of P188 in combination with a DMSO-free composition on the formation of ice and the freezing response of iPS cells.

TABLE 8
Comparison of freezing responses in three different solutions under cooling rate
of −1° C./min and ice nucleation temperature of −4° C.^{a}

| | Solution A | Solution B | Solution C |
|---|---|---|---|
| Measurements | (DMSO-free) | (DMSO-free) | (with DMSO) |
| Post-thaw cell reattachment rate, n = 18 | 104% ± | 48.7% ± | 58.4% ± |
| | 5.73% | 9.85%* | 6.58%* |
| Area fraction of ice in frozen solution, | 76.0% ± | 80.3% ± | 68.6% ± |
| n = 5 | 7.93% | 4.28%^{n.s.} | 10.4%^{n.s.} |
| Distance between adjacent ice crystals | 2.16 ± | 0.670 ± | 1.85 ± |
| (μm), n = 20 | 0.667 | 0.400* | 0.952^{n.s.} |
| Area fraction of intracellular ice in | 2.76% ± | 25.7% ± | 16.6% ± |
| frozen cell aggregate, n ≥ 3 | 1.58% | 23.9%* | 9.05%* |
| Proportion of cells that had intracellular | 0/12 | 6/12* | 5/12* |
| ice, n = 12 | | | |

^{a}Area fraction of ice, distance between adjacent ice crystals, area fraction of intracellular ice and proportion of cells with intracellular ice were quantified from Raman heat maps represented by FIGS. 9D-9F. 95% confidence intervals calculated from samples of size shown for each metric. ANOVA with Bonferroni correction used to determine statistical significance compared to Solution A.
^{n.s.}p > 0.05; *p < 0.05.

In TABLE 9, differential scanning calorimetry results show that the presence of P188 both depressed (by significantly decreasing melting temperature) and suppressed ice formation (by significantly decreasing enthalpy of melting).

TABLE 9
Melting temperatures, enthalpy of melting and glass transition temperatures for
DMSO-free and DMSO CPA solutions measured by DSC^{a}

| CPA solution | Tₘ₁ (° C.) | Tₘ₂ (° C.) | ΔHₘ (J/g) | T_{g} (° C.) |
|---|---|---|---|---|
| Solution A (DMSO-free) | −6.73 ± 0.71 | −2.05 ± 0.09 | 206 ± 21 | −89.2 ± 1.6 |
| Solution A minus albumin | −6.77 ± 0.43 | −1.83 ± 0.29 | 220 ± 13 | −89.6 ± 5.6 |
| Solution A minus isoleucine | −7.03 ± 0.30 | −1.91 ± 0.12 | 212 ± 4 | −89.8 ± 2.4 |
| Solution A minus P188 | −6.26 ± 0.08* | −1.70 ± 0.27* | 230 ± 16* | −91.7 ± 0.7 |
| Solution B (DMSO-free) | −6.62 ± 0.83 | −1.90 ± 0.23 | 211 ± 28 | −88.6 ± 4.9 |
| Solution C (DMSO) | −8.01 ± 0.26* | −2.53 ± 0.12* | 214 ± 9 | −118.5 ± 0.7* |
| P188 alone | −2.90 ± 0.97 | 0.54 ± 0.86 | 254 ± 63 | −69.6 ± 2.2 |

^{a}CPA: cryoprotective agent; DSC: differential scanning calorimetry; Tₘ₁: melting temperature defined as the onset of melting; Tₘ₂: melting temperature defined as the peak of melting; ΔHₘ: enthalpy of melting; T_{g}: glass transition temperature.
Measurements shown as 95% confidence intervals.
Asterisk (*) indicates statistical significance using a two-sample t-test (p < 0.05) compared to Solution A.

### Example 4

### Cell Line and Maintenance of Culture

Induced pluripotent stem (iPS) cell line UMN AMD3-6B4, which was originally derived from conjunctiva of a donor with age-related macular degeneration, was used to develop the cryopreservation method. Cells were cultured in TeSR-E8 media (from STEMCELL Technologies Inc. in Vancouver, Canada) on recombinant human vitronectin (from PeproTech US in Rocky Hill, NJ) and passaged as aggregates using enzyme-free dissociation reagent ReLeSR (STEMCELL Technologies) at a split ratio of 1:8 every 4 days, when colony confluence reached 65-75%.

### Formulation of DMSO-Free Cryopreservation Solution

Basal solution included 5% w/v poloxamer 188 (P188, from Spectrum Chemical in New Brunswick, NJ) in Hank's Balanced Salt Solution with Ca²⁺, Mg²⁺, and glucose (HBSS, from Lonza in Basel, Switzerland). 2× cryopreservation solution contained varying concentrations of trehalose (from Sigma-Aldrich Corporation in St. Louis, MO), glycerol (from Humco in Austin, TX), L-isoleucine (Sigma-Aldrich) and P188 in HBSS. Mixing basal solution and 2× cryopreservation solution at 1:1 volume ratio resulted in diluted solution of trehalose, glycerol, L-isoleucine and P188 in HBSS. The final compositions included:

| Component | Concentration/molarity |
|---|---|
| Trehalose | 13.6-22.9 mM |
| Dextrose | 5.6 mM |
| Glycerol | 410-685 mM |
| L-isoleucine | 10-50 mM |
| Poloxamer 188 | 1-5% w/v |
| CaCl₂ | 1.25 mM |
| MgCl₂ | 0.49 mM |
| MgSO₄ | 0.41 mM |
| KCl | 5.3 mM |
| KH₂PO₄ | 0.44 mM |
| NaHCO₃ | 4.2 mM |
| NaCl | 138 mM |
| Na₂HPO₄ | 0.34 mM |

### Cell Preparation for Cryopreservation

Cells were dissociated using ReLeSR™ and harvested as aggregates without centrifugation in the basal solution at cell concentration of ~3×10⁶/ml. Suspension of cell aggregates was loaded into cryovials (Nunc CryoTube, ThermoFisher Scientific) for 0.5 ml/vial. 2× cryopreservation solution was introduced to the cells dropwise for 0.5 ml/vial. Cells were then incubated at room temperature for 1 hour before freezing.

### Passive Freezing

Cryovials of cells were placed inside a thermos-conductive sample module CoolRack (from Biocision, LLC in San Rafael, CA) that was pre-chilled to 2-8° C. and frozen inside a −20° C. mechanical freezer over a period of 80 minutes. Sample temperature was logged using a DI-245 USB Thermocouple Data Acquisition system (from DATAQ Instruments in Akron, OH). The thermocouple was inserted via a holed, fitted cap into a “dummy” vial containing the same volume of cells and cryopreservation solution as the experimental samples. Frozen cryovials were stored at −20° C.

### Wash-Free Thawing

Frozen cryovials were thawed in a 37° C. water bath. Cryovial was submerged below the lid and agitated for 2.5 min. Thawed suspension of cell aggregates was transferred, without centrifugation or other additives, into TeSR-E8 media using a flow rate of ~1 ml/min. Dilution factor of 2 was used, meaning each cryovial of cells was used to produce 1 well of a 6-well plate of post-thaw cell culture. Diluted cells were seeded onto freshly coated culture vessel and placed in 5% CO₂ at 37° C. incubator undisturbed for 24 h.

### Experimental Results

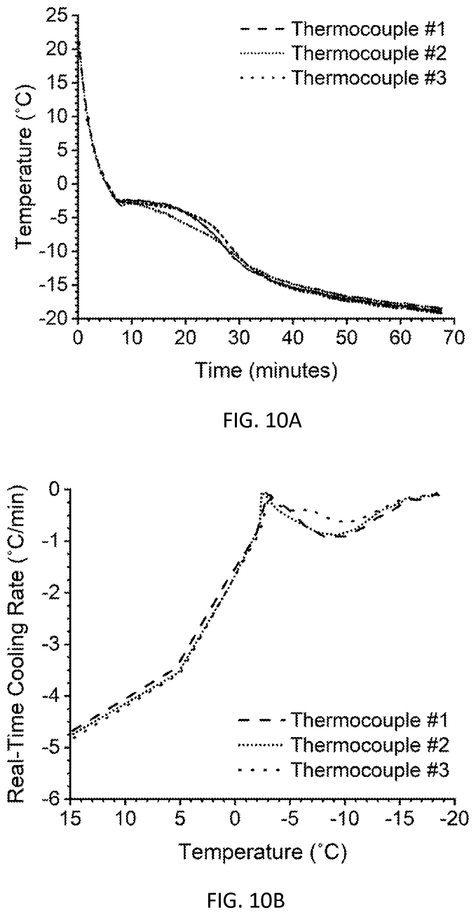

Referring now to FIGS. 10A-10B, the cooling profile of this passive freezing method was assessed. FIG. 10A shows a series of three temperature profiles over time that were largely similar to each other, from three independent replicates of different cryopreservative formulations. FIG. 10B shows a series of three real-time cooling rate profiles corresponding to the temperature changes that were again largely similar to each other, from the same three replicates as above. The real-time cooling rate was the first-order derivative of the sample temperature over time. In other words, slope of the temperature profiles was consistent with y-axis value of the cooling rate profiles. The cooling profile demonstrated by this cryopreservation method can be divided into four stages: first stage being fast cooling of a sample from ambient temperature to just above its freezing point, second stage being ice nucleation and latent heat of fusion reducing cooling rate, third stage being continuous ice growth under a cooling rate close to 1° C./min, and last stage being sample temperature stabilizing near −20° C. This trend of passive freezing using a thermo-conductive sample holder inside a −20° C. freezer resembled that of other common passive freezing methods using an insulated container inside a −80° C. freezer.

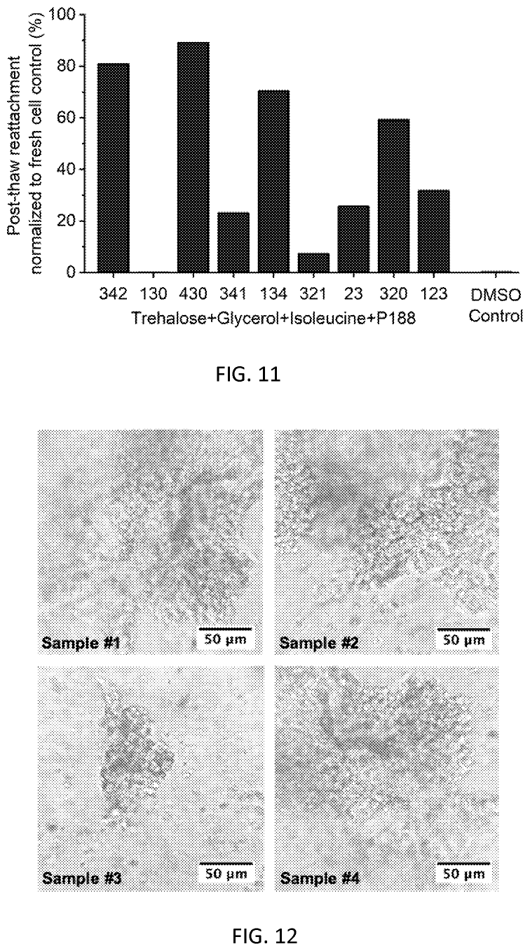

FIG. 11 shows the results of an initial generation of DMSO-free cryopreservative formulations in a differential evolution algorithm-driven development experiment. One set of 9 samples were randomly generated in a parameter space of 4 variable parameters, trehalose, glycerol, isoleucine and P188 that was each discretized into 4 intervals. Post-thaw cell reattachment as the metric was normalized to a fresh cell control. A DMSO control was also used. The DMSO control formulation contained 7.5% v/v DMSO and 5% w/v P188 in HBSS, which was the same formulation as the DMSO control used in Example 3. 8 out of the 9 new formulations exceeded the DMSO control in post-thaw cell reattachment. These formulations contained 13.6-22.9 mM trehalose, 410-685 mM glycerol, 30-50 mM isoleucine, 1-5% w/v P188 and HBSS.

While the DMSO control formulation was feasible in the common controlled-rate freezing and passive freezing methods that cool samples to and store samples at −90° C. or lower, it was not feasible when samples were cooled to and stored at −18° C. or lower and −30° C. or higher. This indicates that cells were significantly more stable in DMSO-free cryopreservative formulation (e.g. formulation #430) and maintained their ability to reattach and survive in post-thaw culture, whereas cells were not stable in the DMSO control formulation and did not maintain their ability to survive in post-thaw culture.

### Example 5

### Cell Line and Maintenance of Culture

The cryopreservation method developed for induced pluripotent stem (iPS) cell line UMN PCBC16iPSV (or vShiPS 9-1) was subsequently tested on committed cardiac progenitor (CCP) cells derived from iPS cell line (hiPSC-CCND2OE). iPS cells were cultured in TeSR-E8 media (from STEMCELL Technologies Inc. in Vancouver, Canada) on hESC-qualified MATRIGEL® (from Corning, Inc. in Corning, NY) and passaged as aggregates using enzyme-free dissociation reagent ReLeSR (STEMCELL Technologies) at a split ratio of 1:8 every 4 days, when colony confluence reached 75-85%. CCPs were obtained as a confluent adherent monolayer in a 12-well plate on day 6 following the 14-day GiWi cardiac differentiation protocol developed by Lian et al.

### Formulation of DMSO-Free Cryopreservation Solution

Basal solution included 5% w/v poloxamer 188 (P188, from Spectrum Chemical in New Brunswick, NJ) in Hank's Balanced Salt Solution with Ca²⁺, Mg²⁺, and glucose (HBSS, from Lonza in Basel, Switzerland). 2× cryopreservation solution included 120 mM sucrose (from Sigma-Aldrich Corporation in St. Louis, MO), 10% v/v glycerol (from Humco in Austin, TX), 15 mM L-isoleucine (Sigma-Aldrich), 5% w/v P188, 4% albumin (ALBUTEIN®, Grifols S.A. in Barcelona, Spain), and 2×MEM non-essential amino acids (NEAA, Sigma-Aldrich) in HBSS. Mixing basal solution and 2× cryopreservation solution at 1:1 volume ratio resulted in 60 mM sucrose, 5% v/v glycerol, 7.5 mM L-isoleucine, 5% w/v P188, 2% albumin, and 1×NEAA in HBSS. The final composition included:

| Component | Concentration/molarity |
|---|---|
| Sucrose | 60 mM |
| Dextrose | 5.6 mM |
| Glycerol | 684 mM |
| L-isoleucine | 7.5 mM |
| Poloxamer 188 | 5% w/v |
| Albumin | 2% w/v |
| CaCl₂ | 1.25 mM |
| MgCl₂ | 0.49 mM |
| MgSO₄ | 0.41 mM |
| KCl | 5.3 mM |
| KH₂PO₄ | 0.44 mM |
| NaHCO₃ | 4.2 mM |
| NaCl | 138 mM |
| Na₂HPO₄ | 0.34 mM |
| Glycine | 0.11 mM |
| Alanine | 0.10 mM |
| Asparagine | 0.10 mM |
| Aspartic acid | 0.10 mM |
| Glutamic acid | 0.10 mM |
| Proline | 0.10 mM |
| Serine | 0.10 mM |

### Cell Preparation for Cryopreservation

Cells were dissociated using 15 mM sodium citrate and harvested as aggregates without centrifugation in the basal solution at cell concentration of ~2×10⁶/ml. Suspension of cell aggregates was loaded into cryovials (Nunc CryoTube, ThermoFisher Scientific) for 0.5 ml/vial. 2× cryopreservation solution was introduced to the cells dropwise for 0.5 ml/vial. Cells were then incubated at room temperature for 1 hour before freezing.

### Controlled-Rate Freezing

Cryovials of cells were frozen using a controlled-rate freezer (Kryo 10 Series III from Planer PLC in Middlesex, UK) following the steps listed below using a cooling rate, B, of 1° C./min and a seeding (or ice nucleation) temperature, T_{NUC}, of −4° C.:
1. Starting temperature 20° C.
2. −10° C./min to 0° C.
3. Hold at 0° C. for 10 min to equilibrate temperature inside and outside cryovials
4. −1° C./min to −4° C.
5. Hold at −4° C. for 15 min
6. Induce ice nucleation when cryovial internal temperature reaches −4° C. towards the end of step 5
7. −1° C./min to −60° C.
8. −10° C./min to −100° C.

Frozen cryovials were transferred in a CRYOPOD Carrier (from Biocision, LLC in San Rafael, CA) and stored in liquid nitrogen.

### Wash-Free Thawing

Frozen cryovials were thawed in a 37° C. water bath. Cryovial was submerged below the lid and agitated for 2.5 min. Thawed suspension of cell aggregates was transferred, without centrifugation or other additives, into RPMI B27 (+) with insulin (from ThermoFisher Scientific in Waltham, MA) using a flow rate of ~1 ml/min. Dilution factor of 2 was used, meaning each cryovial of cells was used to produce 1 well of a 12-well plate of post-thaw cell culture. Diluted cells were seeded onto freshly coated culture vessel and placed in 5% CO₂ at 37° C. incubator undisturbed for 24 h.

### Experimental Results

Distinct from methods commonly used to cryopreserve CCP cells differentiated from iPS cells, the cryopreservation method of the present disclosure maybe performed without apoptosis inhibitors in its cryoprotective formulation or in cell growth media after thawing. Common methods of dissociating CCPs from two-dimensional adherent culture for cryopreservation or subculture purposes use enzymatic reagents such as Accutase (from Innovative Cell Technologies in San Diego, CA), which obtain CCPs in mainly the form of single cells. These single CCP cells would undergo apoptosis without apoptosis inhibition. An enzyme-free chelation agent, sodium citrate, was used instead in the method of the present disclosure to obtain CCPs in mainly the form of multicellular aggregates. FIG. 12 shows brightfield images of CCP aggregates suspended in 5% P188 in HBSS. Majority of cells in the shown regions of interests (ROI) were multicellular aggregates with intact cell-cell contact.

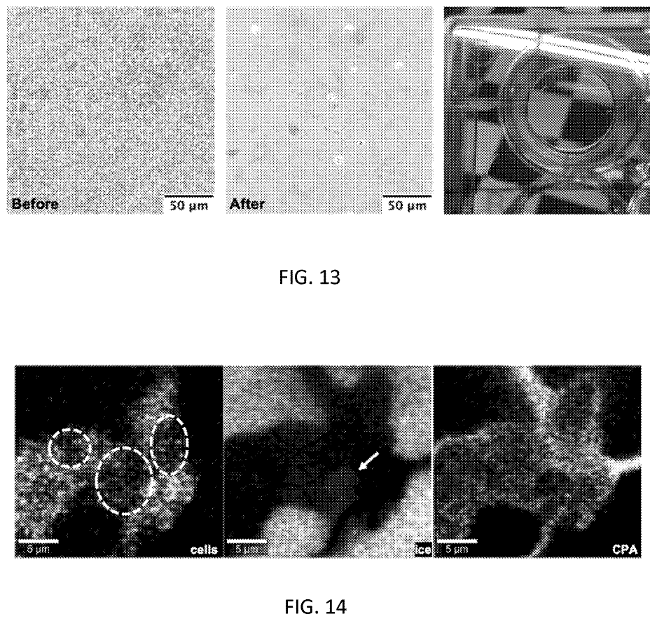

Distinct from difficulty of dissociating entire population of CCPs into suspension in conventional approaches of CCP cryopreservation and subculture, where patches or spots of cells can commonly be visually detected on the culture substrate after the dissociation step, this method resulted in easy detachment and suspension of the entire CCP population upon dissociation. FIG. 13 shows brightfield microscope and cell phone images of the culture substrate covered by a confluent monolayer of CCP cells before the sodium citrate treatment, few to no cells remaining on the culture substrate and the entire bottom of the tissue culture vessel visibly clean after the sodium citrate treatment and P188 suspension.

The cryopreservative formulation used in this Example resulted in the CCP culture at the surface confluence of 67.85% 24 hours after thawing.

FIG. 14 shows heat maps of a frozen CCP cell aggregate and distribution of ice and the cryoprotective agents that were rendered using low-temperature Raman spectroscopy. A mechanism of freezing damage was identified by the intracellular ice formation (arrow) and loss of cell integrity (dashed circles). Partitioning of cryoprotective agent molecules was shown from the significantly higher concentrations in extracellular regions than intracellular regions. The amount of intracellular ice formation observed in these CCPs was much higher than that observed in iPS cells in the same formulation.

### Example 6

### Cell Line and Maintenance of Culture

The cryopreservation method used for induced pluripotent stem (iPS) cell line UMN PCBC16iPSV (or vShiPS 9-1) was subsequently modified to preserve committed cardiac progenitor (CCP) cells derived from iPS cell line (hiPSC-CCND2OE), in the form of confluent adherent monolayers. iPS cells were cultured in TeSR-E8 media (from STEMCELL Technologies Inc. in Vancouver, Canada) on hESC-qualified MATRIGEL® (from Corning, Inc. in Corning, NY) and passaged as aggregates using enzyme-free dissociation reagent ReLeSR (STEMCELL Technologies) at a split ratio of 1:8 every 4 days, when colony confluence reached 75-85%. The 14-day GiWi cardiac differentiation protocol developed by Lian et al was scaled-down to culture cells in 96-well microplates. CCPs were obtained on day 6.

### Formulation of DMSO-Free Cryopreservation Solution

Basal solution included 5% w/v poloxamer 188 (P188, from Spectrum Chemical in New Brunswick, NJ) in Hank's Balanced Salt Solution with Ca²⁺, Mg²⁺, and glucose (HBSS, from Lonza in Basel, Switzerland). 2× cryopreservation solution included varying concentrations of sucrose (from Sigma-Aldrich Corporation in St. Louis, MO), glycerol (from Humco in Austin, TX), L-isoleucine (Sigma-Aldrich) and albumin (ALBUTEIN®, Grifols S.A. in Barcelona, Spain), 5% w/v P188 and 2×MEM non-essential amino acids (NEAA, Sigma-Aldrich) in HBSS. Mixing basal solution and 2× cryopreservation solution at 1:1 volume ratio resulted in diluted solutions of sucrose, glycerol, L-isoleucine, P188, albumin, and NEAA in HBSS. The final composition included:

| Component | Concentration/molarity |
|---|---|
| Sucrose | 20-120 mM |
| Dextrose | 5.6 mM |
| Glycerol | 342-685 mM |
| L-isoleucine | 0-37.5 mM |
| Poloxamer 188 | 5% w/v |
| Albumin | 0-2.5% w/v |
| CaCl₂ | 1.25 mM |
| MgCl₂ | 0.49 mM |
| MgSO₄ | 0.41 mM |
| KCl | 5.3 mM |
| KH₂PO₄ | 0.44 mM |
| NaHCO₃ | 4.2 mM |
| NaCl | 138 mM |
| Na₂HPO₄ | 0.34 mM |
| Glycine | 0.11 mM |
| Alanine | 0.10 mM |
| Asparagine | 0.10 mM |
| Aspartic acid | 0.10 mM |
| Glutamic acid | 0.10 mM |
| Proline | 0.10 mM |
| Serine | 0.10 mM |

### Cell Preparation for Cryopreservation

RPMI B27 (−) without insulin (from ThermoFisher Scientific in Waltham, MA) was removed by aspiration. Basal solution was added to cover the confluent monolayer of cell aggregates for 25 μl/well in a 96-well plate. 2× cryopreservation solution was introduced to the cells slowly for 25 μl/well. Cells were subsequently incubated at room temperature for 1 hour, and 96-well plate was sealed using a silicone well plate liner (from JG Finneran in Vineland, NJ) before freezing.

### Controlled-Rate Freezing

Silicone-sealed plates were frozen using a controlled-rate freezer (Kryo 10 Series III from Planer PLC in Middlesex, UK) following the steps listed below using a cooling rate, B, of 1° C./min and a seeding (or ice nucleation) temperature, T_{NUC}, of −4° C.:
1. Starting temperature 20° C.
2. −10° C./min to 0° C.
3. Hold at 0° C. for 10 min to equilibrate temperature inside and outside cryovials
4. −1° C./min to −4° C.
5. Hold at −4° C. for 15 min
6. Induce ice nucleation when cryovial internal temperature reaches −4° C. towards the end of step 5
7. −1° C./min to −60° C.
8. −10° C./min to −100° C.

Frozen plates were transferred in a CRYOPOD Carrier (from Biocision, LLC in San Rafael, CA) and stored in the vapor phase of liquid nitrogen.

### Wash-Free Thawing

Frozen plates were thawed in a 37° C. water bath. Sealed plate was submerged below the rim of the plate and agitated for 2.5 min. Silicone well liner was removed. RPMI B27 (+) with insulin (from ThermoFisher Scientific in Waltham, MA) was added slowly to the thawed monolayers of cells, for 250 μl/well in the 96-well plate. Diluted cell culture was placed in 5% CO₂ at 37° C. incubator undisturbed for 24 h and subsequently used to resume the 14-day GiWi cardiac differentiation protocol as day-7 cells.

### Experimental Results

A technical foundation was established for high-throughput screening of cryopreservation formulations, or other assays, of cardiac progenitor cells and cardiomyocytes. The GiWi cardiac differentiation protocol was scaled down to 96-well format. On day 16, beating was observed in every well of the 96-well plate that was tested demonstrating successful differentiation. Spread of the cell yield by the confluent monolayers was quantified by staining the cells with Calcein AM. The 95% confidence interval of the cell yield per well of the 96-well plate was measured to be 97.17-102.8% of the mean cell yield per well, demonstrating high consistency in producing the scaled-down cultures of cardiac cell monolayers and suitability of this configuration to quantify post-thaw cell detachment of cryopreserved cell monolayers in experiments such as differential evolution algorithm-driven development of cryopreservative formulation.

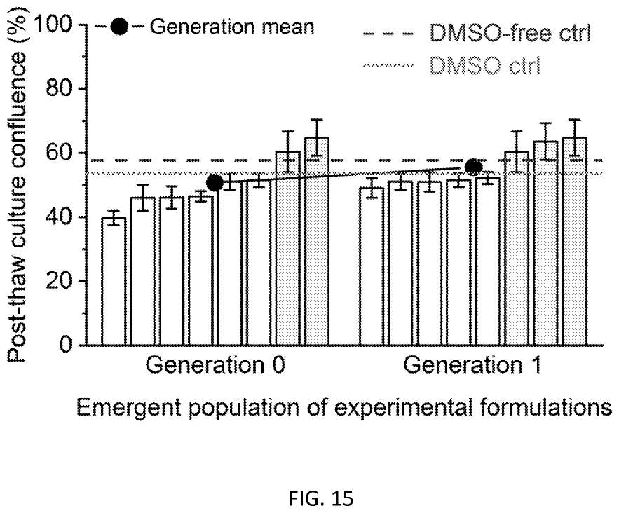

FIG. 15 shows the results of the initial two generations of DMSO-free formulations in a differential evolution algorithm-driven experiment to develop the cryopreservation of CCP monolayers. One set of 8 samples were randomly generated in a parameter space of 4 variable parameters, sucrose, glycerol, isoleucine and albumin that was each discretized into 5 intervals. Post-thaw culture confluence as the metric was normalized to a fresh cell control of a confluent monolayer. A DMSO control was used. The DMSO control formulation contained 7.5% v/v DMSO and 5% w/v P188 in HBSS, which was the same formulation as the DMSO control used in Example 3. A DMSO-free control was also used. The DMSO-free control formulation contained 60 mM sucrose, 5% v/v glycerol, 7.5 mM isoleucine, 2% w/v albumin and 5% w/v P188 in HBSS, which was the same formulation as that developed for iPS cell aggregates in Example 3. Both the generation mean of post-thaw cell survival and the number of experimental formulations that outperformed the DMSO and DMSO-free controls improved from Generation 0 to Generation 1. 3 out of the 16 new formulations exceeded the DMSO control in minimizing cell detachment and keeping majority of the cell monolayer intact in the post-thaw culture. These formulations contained 100-120 mM sucrose, 5% v/v glycerol, 0 mM isoleucine, 0.5-2.5% w/v albumin, 5% w/v P188 and HBSS. Alternative cryoprotective agents may be used such as trehalose. Additional cryoprotective agent may be adjusted as a variable parameter such as P188.

The DMSO-free control formulation developed for controlled-rate freezing of iPS cell aggregates in suspension, which resulted in consistently high post-thaw reattachment rate of ~100%, resulted in 42.3% cell detachment in the cryopreserved monolayer of controlled-rate freezing of adherent CCP monolayers differentiated from iPS cells.

### Experimental Methods Used in the Examples

### Post-Thaw Cell Reattachment

Ability of cryopreserved cells to re-establish culture after thawing was assessed based on a metric of post-thaw cell reattachment. Thawed cells were plated at a specified split ratio. After 24 h, post-thaw culture was washed and stained for live cells with esterase activity using cell permeant dye calcein AM (ThermoFisher Scientific). Live cells in each sample were quantified using a Synergy HT microplate reader (BioTek) in scan mode based on mean fluorescence intensity. Fluorescence measurement was normalized to controls, which were either fresh cells passaged with the same cell density or cells cryopreserved with the same pre-freeze cell concentration using a DMSO-based control formulation, to determine the value of post-thaw cell reattachment for each sample in arbitrary unit.

### Post-Thaw Cell Growth and Colony Formation

Cryopreserved cells were plated at 1:6 thawing ratio. Post-thaw culture of cells cryopreserved in the DMSO-free solution was evaluated label-free by imaging the culture daily with a Cytation 1 cell imaging multi-mode reader (BioTek) with a 4× objective (NA 0.13, Olympus) in the bright-field and scan mode using default focusing method. Images were automatically analyzed by the Gen 5 software (BioTek) using boundary recognition to measure the colony confluence, size and circularity.

### Quality Control of Cryopreserved Cells

Karyotyping was used to detect chromosomal abnormality. Cells were treated with colcemid for 3 h and harvested according to standard cytogenetic protocol. This cell line yielded only 16 metaphase cells, which were completely analyzed by G-banding at a 400 band level resolution.

Expression of surface pluripotency marker, TRA-1-60, was characterized using flow cytometry. Cells were dissociated from culture into single cells using Accutase (from Innovative Cell Technologies, Inc. in San Diego, CA) and stained using mouse anti-TRA-1-60 antibody and Alexa Fluor 488-conjugated goat anti-mouse antibody (ThermoFisher Scientific). Flow cytometry was conducted on an Accuri C6 flow cytometer (from BD Biosciences in San Jose, CA) at low flow rate. 50,000 events were recorded for each sample and gated for forward and side scatter cell population as well as for fluorescence with a negative unstained control.

Expression of transcription factors, OCT-4 and NANOG, were characterized using immunofluorescence and quantitative fluorescence microscopy. Cell culture was fixed using 3.7% formaldehyde (Sigma-Aldrich), permeabilized using 0.2% Triton X-100 (Sigma-Aldrich), and stained using mouse anti-OCT-4 antibody (ThermoFisher Scientific) with Alexa Fluor 488-conjugated goat anti-mouse antibody, goat anti-NANOG antibody (from R&D Systems in Minneapolis, MN) with Alexa Fluor 555-conjugated rabbit anti-goat antibody (ThermoFisher Scientific), against Hoechst 33342 (ThermoFisher Scientific). Images were acquired using a Carl Zeiss Axioskop 50 with a 20× air objective (NA 0.50, Plan-Neofluar, Carl Zeiss) and a Spot Insight Firewire 2 Camera. OCT-4 and NANOG-positive cells were quantified using FIJI by binary threshold and particle analysis.

Trilineage differentiation was performed using STEMdiff Trilineage Differentiation Kit (STEMCELL Technologies) following the directions for use. Differentiated cells of three germ layers were stained for endodermal markers, FOXA2 (from Developmental Studies Hybridoma Bank in Iowa City, IA) and SOX17 (from MilliporeSigma in Burlington, MA), mesodermal markers, NCAM (MilliporeSigma) and T (R&D Systems), and ectodermal markers, PAX6 (R&D Systems) and Nestin (R&D Systems), respectively. Images were acquired using a Cytation 1 Cell Imaging Multi-Mode Reader (from BioTek Instruments, Inc. in Winooski, VT) at 20× magnification.

### Statistics

Mean plus/minus standard error was reported for all measurements unless otherwise noted. Two-tailed student t-tests were performed for two-sample comparisons and one-way ANOVA tests with Bonferroni correction were performed for simultaneous comparisons among multiple samples to obtain p-values with significance level set at 0.05. Null hypothesis was defined as no statistical difference between any pair of samples; p-value less than 0.05 was used to make the decision of rejecting the null hypothesis and determining the significant difference between samples.

### Low-Temperature Raman Spectroscopy

Low-temperature Raman spectroscopy was conducted on a temperature-controlled stage using a four-stage Peltier (Thermonamic Electronics) and a series 800 temperature controller (Alpha Omega Instruments) following the specified controlled-rate freezing steps. At −50° C., Raman spectral data were acquired in 2D scan mode with an integration time of 0.2 s using an Alpha 300R confocal Raman microscope (from WITec in Ulm, Germany) with UHTS300 spectrometer, 600/mm DV401 CCD detector, 532 nm Nd: YAG laser, 100× air objective (NA 0.90 from Nikon), and ~300 nm lateral optical resolution. Raman scattering of crystalline O—H stretching centered at wavenumber of 3125 cm⁻¹ was used to characterize ice; that of amide I and C═C stretching at 1660 cm⁻¹ was used to characterize cells; that of CCO rocking at 485 cm⁻¹ was used to characterize glycerol; that of CH₂ twisting at 1285 cm⁻¹ was used to characterize P188; that of CC stretching at 850 cm⁻¹ was used to characterize all DMSO-free CPA molecules; and that of symmetric CS stretching at 673 cm⁻¹ was used to characterize DMSO. Raman spectra were acquired of each sample using a constant laser power over an integration time of 5 s and averaged over 10 accumulations. Raman heat maps of the specified substances were each rendered from the area under peak at its characteristic Raman wavenumber at 3 pixels/μm to visualize its distribution and their spatial relationship with each other.

### Development of Buffer and Additives

Controlled-rate freezing protocol remained constant with a cooling rate of 1° C./min and an ice nucleation temperature of −4° C. Composition of osmolytes (i.e., 30 mM sucrose, 5% v/v glycerol, 7.5 mM isoleucine in Normosol R) was adapted from the cryopreservation solution previously optimized for hMSC (see Pollock K., Algorithm optimization of cryopreservation protocols to improve mesenchymal stem cell functionality. Dissertation, University of Minnesota, 2016. Available at hdl.handle.net/11299/191466), a fundamental formulation that was used as a starting point.

Different forms of cellular damage were identified. Low-temperature spectroscopy was conducted as described earlier to characterize the formation of intracellular ice at −50° C. Both pre-freeze and post-thaw, cells were stained for ROS and free radicals using H2DCFDA (ROS Detection Assay Kit, BioVision) at 37° C. for 45 min to characterize oxidative stress by immunofluorescence and microplate assay. Thawed cells were also stained for caspase enzyme activity using FAM-FLICA (from ImmunoChemistry Technologies, LLC in Bloomington, MN) to detect apoptosis. Size of cell aggregates post-thaw was observed visually. Post-thaw cell recovery, reattachment and proliferation were measured as described earlier. Cells cryopreserved using 10% DMSO in PBS++ was used as a control. Brightfield and fluorescent images were acquired using a Carl Zeiss Axioskop 50 with a 10× air objective (NA 0.30, Plan-Neofluar, Carl Zeiss) or a 20× air objective (NA 0.50, Plan-Neofluar, Carl Zeiss) and a Spot Insight Firewire 2 Camera. Microplate assay was quantified using a Synergy HT microplate reader (BioTek).

Different types of additives were tested. P188 5% w/v was added to both the basal solution and the 2× cryopreservation solution to stabilize cell membrane. 2×NEAA was added pre-freeze to the 2× cryopreservation solution to conserve cellular energy. The following molecules were used to target general or specific apoptosis pathways and added at the given working concentration either pre-freeze or post-thaw: 10 μM rho kinase inhibitor (or ROCK inhibitor, Y-27632, STEMCELL Technologies), 20 μM pan caspase inhibitor (Z-VAD-FMK, from ApexBio Technology LLC in Houston, TX), 1 μg/ml Fas ligand inhibitor (Recombinant Human Fas/TNFRSF6-Fc Chimera from BioLegend in San Diego, CA), 100 ng/ml each of TNF-related apoptosis-inducing ligand (TRAIL) inhibitors targeting TRAIL receptor 1 (Recombinant Human TNFRSF10A-Fc Chimera, BioLegend), TRAIL receptor 2 (Recombinant Human TNFRSF10B-Fc Chimera, BioLegend), and TRAIL receptor 4 (Recombinant Human TNFRSF10D-Fc Chimera, BioLegend) respectively.

Two different types of buffer solutions were tested to replace Normosol R. Phosphate-buffered saline with Mg²⁺ and Ca²⁺ (PBS++, in-house) was compared to Hank's Balanced Salt Solution with Mg²⁺, Ca²⁺, HCO₃⁻ and glucose (HBSS, Lonza) as a part of both the basal solution and 2× cryopreservation solution. They were intended to maintain salt balance and neutral pH under ambient atmospheric condition in pre-freeze and post-thaw cell processing. HEPES was not considered as a buffer for the basal or cryopreservation solution, and upon wash-free thawing, HEPES contained in E8 would play the role of maintaining neutral pH under 5% CO₂ condition.

### Screening of Cryopreservation Solution

Different types of sugars (disaccharides) and sugar alcohols (all BioUltra or PharmaGrade, Sigma-Aldrich) were individually tested for cytotoxicity limits based on its effect on cell reattachment. In consideration for pre-freeze cell processing, each molecule of a given concentration among 300 mM, 150 mM, and 75 mM was used to incubate cell aggregates suspended in HBSS at room temperature for 30 minutes or 1 hour. Then, in consideration of wash-free thawing at minimum dilution ratio of 2, each sample of cell aggregates were mixed at 1:1 volume ratio with E8 media and plated on vitronectin-coated culture vessel. 24 hours later, cell reattachment was observed qualitatively under a Nikon TMS inverted phase contrast microscope. Disaccharides included sucrose, trehalose, maltose, and lactose. Sugar alcohols included ethylene glycol, glycerol (Humco), D-Sorbitol, D-mannitol, xylitol, myo-inositol, and adonitol.

### Differential Evolution Algorithm-Driven Optimization of Cryopreservation Formulation

A DE algorithm with the basic mutation strategy (DE/rand/1/bin) (Storn and Price, 1997) was used to rapidly optimize the composition of the DMSO-free freezing solution for hiPSCs based on the functional metric of post-thaw reattachment rate. Briefly, the DE algorithm utilizes stochastic direct search to randomly generate an initial group (Generation 0) of sample parameters (i.e. concentrations of DMSO-free CPA molecules) from the population spanning the entire parameter space. Generation 0 samples were tested experimentally, and their post-thaw reattachment rates were used by the algorithm to output the next group (Generation 1) of CPA concentrations that were mutated versions of Generation 0 to be tested. Cumulatively best members of each generation were stored as an emergent population. The algorithm-driven optimization was completed, and convergence was achieved when emergent population of the latest generation was the same as that of the previous generation. Parameter space of the DE algorithm was determined by the cytotoxicity limit and was discretized into multiple intervals. Cytotoxicity was defined as decrease in cell reattachment in the cell culture measured using calcein AM 24 hours after 1-hour exposure to the respective molecules compared to fresh cell control.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A cryopreservative composition comprising:
a sugar component, wherein total concentration of the sugar component in the composition is 300 mM or less;
a sugar alcohol component, wherein total concentration of the sugar alcohol component in the composition is 2 M or less; and
an amino acid component at a concentration of 0.1 mM to 100 mM, wherein the amino acid component comprises glycine, alanine, asparagine, aspartic acid, glutamic acid, proline, and serine;
with the proviso that the composition comprises less than 5% (w/v) dimethyl sulfoxide (DMSO).

2. The cryopreservative composition of claim 1, wherein the sugar component is provided at a concentration of 1 mM to 250 mM, from 10 mM to 200 mM, from 20 to 120 mM, from 25 to 100 mM, or from 30 mM to 80 mM.

3. The cryopreservative composition of claim 1, wherein the sugar component comprises trehalose, maltose, lactose, fructose, sucrose, glucose, dextran, melezitose, raffinose, nigerotriose, maltotriose, maltotriulose, kestose, cellobiose, chitobiose, lactulose, or a combination thereof.

4. The cryopreservative composition of claim 1, wherein the sugar component comprises from 10 mM to 200 mM, from 20 to 120 mM, or from 30 mM to 80 mM of trehalose, sucrose, maltose, lactose, or a combination thereof.

5. The cryopreservative composition of claim 1, wherein the sugar alcohol component is provided at a concentration of 0.2 M to 1.2 M, 0.2 M to 1 M, or 0.3 M to 0.8 M.

6. The cryopreservative composition of claim 1, wherein the sugar alcohol component comprises glycerol, sorbitol, ethylene glycol, propylene glycol, inositol, xylitol, mannitol, arabitol, ribitol, erythritol, threitol, galactitol, pinitol, or a combination thereof.

7. The cryopreservative composition of claim 1, comprising 0.2 M to 1.0 M glycerol.

8. The cryopreservative composition of claim 1, further comprising a polymer component at a concentration of no more than 15% (w/v).

9. The cryopreservative composition of claim 8, comprising at least 1% (w/v), at least 2%, at least 3%, at least 4%, or at least 5% of the polymer component and no more than 12% (w/v), no more than 10%, no more than 8%, no more than 7%, no more than 6%, or no more than 5% of the polymer component, wherein the polymer component comprises poloxamer.

10. The cryopreservative composition of claim 1, further comprising albumin at a concentration of 0.5% to 10% (w/v).

11. The cryopreservative composition of claim 1, further comprising an ionic component at a concentration of at least 0.05% (w/v), at least 0.1%, at least 0.2%, at least 0.3%, at least 0.4%, at least 0.5%, at least 0.6%, or at least 0.7%, and no more than 2.5% (w/v), no more than 2%, no more than 1.5%, no more than 1.3%, no more than 1.2%, no more than 1.1%, or no more than 1.0%, wherein the ionic component comprises salt, acid, base or a combination thereof.

12. The cryopreservative composition of claim 11, wherein the ionic component comprises a balanced salt solution.

13. The cryopreservative composition of claim 11, wherein the ionic component comprises CaCl₂, MgCl₂, MgSO₄, KCl, KH₂PO₄, NaHCO₃, NaCl, and Na₂HPO₄.

14. The cryopreservative composition of claim 1, wherein the amino acid component further comprises isoleucine, creatine, lysine, sarcosine, or a combination thereof.

15. The cryopreservative composition of claim 1, further comprising adenosine, guanidine, or a combination thereof.

16. The cryopreservative composition of claim 1, further comprising a secondary amino acid component comprising one or more amino acids, amino acid derivatives, peptides, or a combination thereof.

17. The cryopreservative composition of claim 16, wherein the secondary amino acid component comprises proline, valine, alanine, glycine, asparagine, aspartic acid, glutamic acid, serine, histidine, cysteine, tryptophan, tyrosine, arginine, glutamine, taurine, betaine, ectoine dimethylglycine, ethylmethylglycine, an RGD peptide, or a combination thereof.

18. The cryopreservative composition of claim 1, further comprising a cell.

19. The cryopreservative composition of claim 18, wherein the cell is an iPS cell, an embryonic stem cell, a cardiac progenitor cell, a cardiomyocyte, a neural progenitor cell, a neuron, a glial cell, a beta cell, an endothelial cell, an epithelial cell, a smooth muscle cell, a tenocyte, an osteocyte, a chondrocyte, an adipocyte, a corneal cell, a retinal cell, a trabecular meshwork cell, an intestinal cell, a renal cell, a hematopoietic cell, a gamete, or a combination thereof.

20. The cryopreservative composition of claim 18, wherein the cell is viable or functional for its intended use when recovered from cryopreservation.

21. A method of cryopreserving a cell, the method comprising:
adding a cell to the cryopreservative composition of claim 1; and
freezing the composition.

22. The method of claim 21, wherein the freezing of the composition comprises initiating crystallization of molecular components in the cryopreservative composition at an ice nucleation temperature, wherein the ice nucleation temperature is 0° C. to −3° C., −1° C. to −20° C., −1° C. to −12° C., −12° C. to −20° C., −6° C. to −12° C., −1° C. to −8° C., −1.5° C. to −7° C., −2° C. to −6° C., −3° C. to −6° C., or −3° C. to −5° C.

23. The method of claim 21, wherein the freezing of the composition comprises cooling at a rate of 0.1° C./min to 5° C./min, 0.3° C./min to 3° C./min, or 0.8° C./min to 1.2° C./min.

24. The method of claim 21, further comprising thawing and using the cryopreservative composition with or without a washing step.
